(19) United States
(12) Patent Application Publication
(10) Pub. No.: US 2015/0164895 A1
(43) Pub. Date: Jun. 18, 2015

(54) DETERMINING SENSITIVITY OF CELLS TO B-RAF INHIBITOR TREATMENT BY DETECTING KRAS MUTATION AND RTK EXPRESSION LEVELS

(71) Applicant: GENENTECH, INC., SOUTH SAN FRANCSICO, CA (US)

(72) Inventors: Georgia Hatzivassiliou, San Francisco, CA (US); Shiva Malek, San Francisco, CA (US)

(73) Assignee: GENENTECH, INC., SOUTH SAN FRANCSICO, CA (US)

(21) Appl. No.: 14/284,027

(22) Filed: May 21, 2014

Related U.S. Application Data
(63) Continuation of application No. 13/392,405, filed on May 4, 2012, now abandoned.
(60) Provisional application No. 61/236,466, filed on Aug. 24, 2009.
(60) Provisional application No. 61/301,149, filed on Feb. 3, 2010.

(51) Int. Cl. A61K 31/517; G01N 33/574

(52) U.S. Cl. CPC A61K 31/517; G01N 33/57492; G01N 2333/914; G01N 2333/91205

(57) ABSTRACT

The present invention relates to prognostic methods for identifying tumors that are not susceptible to B-Raf inhibitor treatment by detecting mutations in a K-ras gene or protein or by detecting overexpression of RTKs and/or their ligands. Kits are also disclosed for carrying out the methods.

### CROSS-REFERENCE TO RELATED APPLICATIONS

This is a Continuation application of U.S. application Ser. No. 13/392,405, filed May 4, 2012, which is a 371 of International Application No. PCT/US2010/46520, filed Aug. 24, 2010 which claims the benefit under 35 U.S.C. §119(e) of U.S. Provisional Application Ser. Nos. 61/236,466 filed Aug. 24, 2009 and 61/301,149 filed Feb. 3, 2010, which are incorporated herein by reference in their entirety for all purposes.

### FIELD OF THE INVENTION

The present invention relates to cancer diagnostics and therapies and in particular to the detection of mutations or RTK overexpression that are diagnostic and/or prognostic and correlating the detection with cancer treatment.

### BACKGROUND OF INVENTION

Receptor tyrosine kinases (RTKs) and their ligands are important regulators of tumor cell proliferation, angiogenesis, and metastasis. For example, the ErbB family of RTKs include EGFR (HER1 and ErbB1), HER2 (neu or ErbB2), HER3 (ErbB3), and HER4 (ErbB4), and have distinct ligand-binding and signaling activities. Ligands that bind to ErbB receptors include epidermal growth factor (EGF), transforming growth factor a (TGFa), heparin-binding EGF-like ligand (HB-EGF), amphiregulin (AR), betacellulin (BTC), epiregulin (EPR), epigen (EPG), heregulin (HRG), and neuregulin (NRG). These ligands bind directly to EGFR, HER3, or HER4 and trigger multiple downstream signaling cascades, including the RAS-ERK and PI3K-Akt pathways. EGF and other growth factors and cytokines, such as platelet-derived growth factor (PDGF), signal via Ras. Ras mutations permanently lock Ras in its active, GTP-bound state (Wislez, M, et al., Cancer Drug Discovery and Development: EGFR Signaling Networks in Cancer Therapy, Eds: J. D. Haley and W. J. Gullick, Humana Press, pp. 89-95, 2008).

MET is another RTK whose activation by its ligand hepatocyte growth factor (HGF) induces MET kinase catalytic activity, which triggers transphosphorylation of the tyrosines Tyr 1234 and Tyr 1235. These two tyrosines engage various signal transducers, thus initiating a whole spectrum of biological activities driven by MET. HGF induces sustained RAS activation, and thus prolonged MAPK activity.

K-ras is one of ras genes that undergo mutation in various cancers. The mutation of the K-ras gene at codons 12 and 13 takes part in tumorigenesis which leads to functional modification of p21-ras protein, a K-ras gene product, resulting in transferring excessive growth signals to a cell nuclei to stimulate cell growth and division. Therefore, identification of mutations of K-ras gene has been widely used as a useful tool in cancer diagnosis, e.g., pancreatic, colorectal and non-small cell lung cancers, and studies have suggested that it might be associated with some tumor phenotypes (Samowitz W S, et al., Cancer Epidemiol. Biomarkers Prev. 9: 1193-1197, 2000; Andreyev H J, et al., Br. J. Cancer 85: 692-696, 2001; and Brink M, et al., Carcinogenesis 24: 703-710, 2003).

Ras plays an essential role in oncogenic transformation and genesis. Oncogenic H-, K-, and N-Ras arise from point mutations limited to a small number of sites (amino acids 12, 13, 59 and 61). Unlike normal Ras, oncogenic ras proteins lack intrinsic GTPase activity and hence remain constitutively activated (Trahey, M., and McCormick, F. (1987) Science 238: 542-5; Tabin, C. J. et al. (1982) Nature. 300: 143-9; Taparowsky, E. et al. (1982) Nature. 300: 762-5). The participation of oncogenic ras in human cancers is estimated to be 30% (Almoguera, C. et al (1988) Cell. 53:549-54).

Mutations are frequently limited to only one of the ras genes, and the frequency is tissue- and tumor type-specific. K-ras is the most commonly mutated oncogene in human cancers, especially the codon-12 mutation. While oncogenic activation of H-, K-, and N-Ras arising from single nucleotide substitutions has been observed in 30% of human cancers (Bos, J. L. (1989) Cancer Res 49, 4682-9), over 90% of human pancreatic cancer manifest the codon 12 K-ras mutation (Almoguera, C. et al. (1988) Cell 53, 549-54; Smit, V. T. et al. (1988) Nucleic Acids Res 16, 7773-82; Bos, J. L. (1989) Cancer Res 49, 4682-9). Pancreatic ductal adenocarcinoma, the most common cancer of the pancreas, is notorious for its rapid onset and resistance to treatment. The high frequency of K-ras mutations in human pancreatic tumors suggests that constitutive Ras activation plays a critical role during pancreatic oncogenesis. Adenocarcinoma of the exocrine pancreas represents the fourth-leading cause of cancer-related mortality in Western countries. Treatment has had limited success and the five-year survival remains less than 5% with a mean survival of 4 months for patients with surgically unresectable tumors (Jemal, A et al (2002) CA Cancer J Clin 52, 23-47; Burris, H. A., 3rd et al. (1997) J Clin Oncol 15, 2403-13). This point mutation can be identified early in the course of the disease when normal cuboidal pancreatic ductal epithelium progresses to a flat hyperplastic lesion, and is considered causative in the pathogenesis of pancreatic cancer (Hruban, R. H. et al (2000) Clin Cancer Res 6, 2969-72; Tada, M. et al. (1996) Gastroenterology 110, 227-31). The regulation of oncogenic K-ras signaling in human pancreatic cancer, however, remains largely unknown.

K-ras mutations are present in 50% of the cancers of colon and lung (Bos, J. L. et al. (1987) Nature. 327: 293-7; Rodenhuis, S. et al. (1988) Cancer Res. 48: 5738-41). In cancers of the urinary tract and bladder, mutations are primarily in the H-ras gene (Fujita, J. et al. (1984) Nature. 309: 464-6; Visvanathan, K. V. et al. (1988) Oncogene Res. 3: 77-86). N-ras gene mutations are present in 30% of leukemia and liver cancer. Approximately 25% of skin lesions in humans involve mutations of the Ha-Ras (25% for squamous cell carcinoma and 28% for melanomas) (Bos, J. L. (1989) Cancer Res. 49:4683-9; Migley, R. S, and Kerr, D. J. (2002) Crit Rev Oncol Hematol. 44:109-20). 50-60% of thyroid carcinomas are unique in having mutations in all three genes (Adjei, A. A. (2001) J Natl Cancer Inst. 93: 1062-74).

Constitutive activation of Ras can be achieved through oncogenic mutations or via hyperactivated growth factor receptors such as the EGFRs. Elevated expression and/or amplification of the members of the EGFR family, especially the EGFR and HER2, have been implicated in various forms of human malignancies (as reviewed in Prenzel, N. et al. (2001) Endocr Relat Cancer. 8: 11-31). In some of these cancers (including pancreas, colon, bladder, lung), EGFR/HER2 overexpression is compounded by the presence of oncogenic Ras mutations. Abnormal activation of these receptors in tumors can be attributed to overexpression, gene amplification, constitutive activation mutations or autocrine growth factor loops (Voldborg, B. R. et al. (1997) Ann Oncol. 8: 1197-206). For growth factor receptors, especially the EGFRs, amplification or/and overexpression of these receptors frequently occur in the cancers of the breast, ovary, stomach, esophagus, pancreatic, lung, colon and neuroblastoma.

The RAS-MAPK signaling pathway controls cell growth, differentiation and survival. This signaling pathway has long been viewed as an attractive pathway for anticancer therapies, based on its central role in regulating the growth and survival of cells from a broad spectrum of human tumors, and mutations in components of this signaling pathway underlie tumor initiation in mammal cells (Sebolt-Leopold et al (2004) Nat Rev Cancer 4, pp 937-47).

The RAS-MAPK signaling pathway is activated by a variety of extracellular signals (hormones and growth factors), which activate RAS by exchanging GDP with GTP. Ras then recruits RAF to the plasma membrane where its activation takes place. As noted above, mutations in components of the signaling pathway, resulting in constitutive activation, underlie tumor initiation in mammalian cells. For example, growth factor receptors, such as epidermal growth factor receptor (EGFR), are subject to amplifications and mutations in many cancers, accounting for up to 25% of non-small cell lung cancers and 60% of glioblastomas. Braf is also frequently mutated, particularly in melanomas (approximately 70% of cases) and colon carcinomas (approximately 15% of cases). Moreover, ras is the most frequently mutated oncogene, occurring in approximately 30% of all human cancers. The frequency and type of mutated ras genes (H-ras, K-ras or N-ras) varies widely depending on the tumor type. K-ras is, however, the most frequently mutated gene, with the highest incidence detected in pancreatic cancer (approximately 90%) and colorectal cancer (approximately 45%). This makes it, as well as other components of the signaling pathway, an appropriate target for anticancer therapy. Indeed, small-molecular weight inhibitors designed to target various steps of this pathway have entered clinical trials. Moreover, sorafenib (Nexavar®, Bayer HealthCare Pharmaceuticals), a RAF-kinase inhibitor resulting in RAS signaling inhibition, has recently been approved against renal cell carcinoma. Following these data, there continues to be a high level of interest in targeting the RAS-MAPK pathway for the development of improved cancer therapies.

The RAS-MAPK signaling pathway is activated by a variety of extracellular signals (hormones and growth factors), which activate RAS by exchanging GDP with GTP. Ras then recruits RAF to the plasma membrane where its activation takes place. As noted above, mutations in components of the signaling pathway, resulting in constitutive activation, underlie tumor initiation in mammalian cells. For example, growth factor receptors, such as epidermal growth factor receptor (EGFR), are subject to amplifications and mutations in many cancers, accounting for up to 25% of non-small cell lung cancers and 60% of glioblastomas. Braf is also frequently mutated, particularly in melanomas (approximately 70% of cases) and colon carcinomas (approximately 15% of cases). Moreover, ras is the most frequently mutated oncogene, occurring in approximately 30% of all human cancers. The frequency and type of mutated ras genes (H-ras, K-ras or N-ras) varies widely depending on the tumor type. K-ras is, however, the most frequently mutated gene, with the highest incidence detected in pancreatic cancer (approximately 90%) and colorectal cancer (approximately 45%). This makes it, as well as other components of the signaling pathway, an appropriate target for anticancer therapy. Indeed, small-molecular weight inhibitors designed to target various steps of this pathway have entered clinical trials. Moreover, sorafenib (Nexavar®, Bayer HealthCare Pharmaceuticals), a RAF-kinase inhibitor resulting in RAS signaling inhibition, has recently been approved against renal cell carcinoma. Following these data, there continues to be a high level of interest in targeting the RAS-MAPK pathway for the development of improved cancer therapies.

As described in Downward, J. (2002) Nature Reviews Cancer, volume 3, pages 11-22, the RAS proteins are members of a large superfamily of low-molecular-weight GTP-binding proteins, which can be divided into several families according to the degree of sequence conservation. Different families are important for different cellular processes. For example, the RAS family controls cell growth and the RHO family controls the actin cytoskeleton. Conventionally, the RAS family is described as consisting of three members H-, N- and K-RAS, with K-RAS producing a major (4B) and a minor (4A) splice variant (Ellis, C. A and Clark, G. (2000) Cellular Signalling, 12:425-434). The members of the RAS family are found to be activated by mutation in human tumors and have potent transforming potential.

The RAS members are very closely related, having 85% amino acid sequence identity. Although the RAS proteins function in very similar ways, some indications of subtle differences between them have recently come to light. The H-ras, K-ras and N-ras proteins are widely expressed, with K-ras being expressed in almost all cell types. Knockout studies have shown that H-ras and N-ras, either alone or in combination, are not required for normal development in the mouse, whereas K-ras is essential (Downward, J. (2002) at page 12).

Furthermore, as described in Downward, J. (2002), aberrant signaling through RAS pathways occurs as the result of several different classes of mutational damage in tumor cells. The most obvious of these mutations is in the ras genes themselves. Some 20% of human tumors have activating point mutations in ras, most frequently in K-ras (about 85% of total), then N-ras (about 15%), then H-ras (less than 1%). These mutations all compromise the GTPase activity of RAS, preventing GAPs from promoting hydrolysis of GTP on RAS and therefore causing RAS to accumulate in the GTP-bound, active form. Almost all RAS activation in tumors is accounted for by mutations in codons 12, 13 and 61 (Downward, J. (2002) at page 15).

It would be useful if cancer treatment could be tailored to the specific cancer. In particular, the present invention provides for a means of determining whether certain approved and available treatments would nevertheless not be of benefit for the particular type of cancer.

### BRIEF SUMMARY OF INVENTION

The present invention relates to prognostic methods for identifying tumors that are not susceptible to B-Raf inhibitor treatment by detecting mutations in a K-ras gene or protein. The methods involve determining the presence or absence of a mutated K-ras gene or protein in a sample thereby identifying a tumor that is non-responsive to B-Raf inhibitor treatment. Kits are also disclosed for carrying out the methods.

In another aspect, the present invention relates to prognostic methods for identifying tumors that are not susceptible to B-Raf inhibitor treatment by detecting aberrant expression levels of RTKs. The methods involve determining the expression levels of certain RTKs in a sample, whereby overexpression of RTKs correlate non-responsiveness to B-Raf inhibitor treatment. Examples of RTKs that correlate to the responsiveness of B-Raf treatment include, but are not limited to EGFR and cMet. The methods also involve determining the induction levels of certain ligands of RTKs in a sample whereby abnormally high levels of ligand induction correlates with non-responsiveness to B-Raf inhibitor treatment. Examples of ligands that correlate to the responsiveness of B-Raf treatment include, but are not limited to EGF and HGF. The methods also involve determining the levels of Ras-GTP in a sample whereby abnormally high levels of Ras-GTP correlates with non-responsiveness to B-Raf inhibitor treatment. Kits are also disclosed for carrying out the methods.

In another aspect, the present invention relates to methods of treating a tumor that is non-responsive to B-Raf inhibitor treatment. The methods include administering a B-Raf inhibitor in combination with an EGFR inhibitor.

### DESCRIPTION OF THE FIGURES

FIG. 1 depicts Biochemical enzyme assay data. The data show that at physiological [ATP], only GDC-0879 maintains effective potency against both B-Raf^{V600E} and WT Raf isoforms.

FIG. 2 depicts Viability assays in tumor lines of different Raf/Ras mutational status.

FIG. 3 depicts sustained pMEK induction by Raf inhibitors only in non-B-Raf^{V600E} lines. pMEK levels plateau relatively to inhibitors' IC₅₀ against WT Raf.

FIG. 4 depicts c-Raf is the Raf isoform primarily responsible for the pMEK induction by Raf inhibitors in non B-Raf^{V600E} lines.

FIG. 5 depicts c-Raf specific activity induced by both inhibitors only in non-B-Raf^{V600E} lines. There was no decrease in Sprouty levels under conditions of Raf induction.

FIG. 6 depicts no induction of pERK levels. Inhibitors' relative potencies correlate with their biochemical IC₅₀s.

FIG. 7 depicts bell-shaped effects on pMEK levels under basal conditions. Inhibitory effects of GDC-0879 predominate after serum stimulation.

FIG. 8A depicts the duration and extent of BRAF pathway inhibition determines B-Raf inhibitor, GDC-0879 efficacy in primary human tumor xenograft models. A Kaplan-Meier plot showing time to tumor doubling for patient-derived melanoma and non-small cell lung cancer tumor models treated daily with 100 mg/kg GDC-0879 or vehicle. Genotypes for BRAF, N-ras and K-ras are indicated. A statistically significant (P<0.05) delay in tumor progression was noted for MEXF 989, MEXF 276, and MEXF 355 tumors. GDC-0879 administration significantly accelerated growth of some K-ras-mutant non-small cell lung tumors, such as LXFA 1041 and LXFA 983.

FIG. 8B depicts GDC-0879 treatment down-regulated ERK1/2 phosphorylation in BRAF^{V600E} primary human xenograft tumors. In time course pharmacodynamic studies, mice were treated with 100 mg/kg GDC-0879 and scarified at 1 or 8 h following the last dose (days 21-24). Immunoblots of phosphorylated and total ERK1/2 are shown. Potent phosphor-ERK1/2 inhibition sustained through 8 h was strongly correlated with BRAF^{V600E} status and GDC-0879 antitumor efficacy. Total ERK1/2 expression was examined in all samples as a loading control.

FIGS. 9A, B, C & D depict K-ras-mutant tumor cell lines show differential sensitivity to GDC-0879 RAF and MEK inhibitors in vivo and in vitro. A and B, inhibition of MEK, but not RAF, prevented the in vivo growth of K-RAS-mutant HCT116 tumors. Mice were randomized when tumors reached ˜200 mm³ and treatment was initiated with either 100 mg/kg GDC-0879 (A) or 25 mg/kg MEK inhibitor (MEK Inh; B) on a daily schedule. Points, mean; bars, SE. C, GDC-0879 EC₅₀ values for 130 cell lines are shown as a function of BRAF and K-RAS mutational status. GDC-0879-mediated inhibition of cell growth was strongly correlated with BRAF mutation. D, dot plots for MEK inhibitor EC₅₀ values are organized according to genotype. MEK inhibition was also potent on a significant fraction of cell lines expressing wild-type BRAF. Data represents the mean of quadruplicate measurements.

FIGS. 10-18 depict growth in lung tumor xenografts after dosing with GDC-0879.

FIGS. 19A and B depict Raf inhibitors inducing RAS-dependent translocation of wildtype RAF to the plasma membrane in non-B-RAFV600E cells. (A) MeWo (RAS/RAFWT) cells were treated with GDC-0879 (2-{4-[(1E)-1-(hydroxyimino)-2,3-dihydro-1H-inden-5-yl]-3-(pyridine-4-yl)-1H-pyrazol-1-yl}ethan-1-ol), PLX4720 (N-[3-[(5-chloro-1H-pyrrolo[2,3-b]pyridin-3-yl)carbonyl]-2,4-difluorophenyl]-1-propanesulfonamide) or AZ-628 (3-(2-cyanopropan-2-yl)-N-(4-methyl-3-(3-methyl-4-oxo-3,4-dihydroquinazolin-6-ylamino)phenyl)benzamide) (all at 0.1, 1, 10 mM) for 1 hr and fractionated into membrane (P100) and cytosolic (S100) fractions. Aliquots of the membrane and cytosolic fractions were immunoblotted with the indicated antibodies. (B) HEK293T cells were transiently transfected with Venus-C-RAF (green), CFP-K-RAS (red) and mCherry-H2B (blue). Venus-tagged C-RAF co-localizes with CFP-KRAS on the plasma membrane in cells treated with 10 mM GDC-0879 or AZ-628 for 4 hours followed by live cell imaging using confocal fluorescence microscopy. Membrane translocation is blocked when the dominant negative CFP-tagged KRASSI7N is transfected instead of KRASWT (right panel).

FIGS. 20A, B, C and D depict the importance of the role of active Ras plays in C-RAF activation and phospho-MEK induction by RAF inhibitors. (A) A375 (B-RAFV600E) cells were treated with GDC-0879 or PLX4720 for 1 hour and lysed in hypotonic buffer for membrane fractionation. Both membrane (P100) and cytosolic (S100) fractions were immunoblotted with the indicated antibodies. (B) MeWo cells were transiently transfected with KRASWT or KRASSI7N, treated with GDC-0879 or PLX4720 (at 0.1, 1, 10 mM) for 1 hour and fractionated into membrane (P100) and cytosolic (S100) fractions. Aliquots of the membrane and cytosolic fractions were immunoblotted with anti-phospho- and anti-total MEK antibodies. (C) RAS-GTP levels were measured from lysates of MeWo (RAS/RAFWT), A375 (B-RAFV600E) and H2122 (KRASMT) cells with a Ras-GTP ELISA protocol using immobilized C-RAF-RBD as bait for capturing RAS-GTP. Relative luminescent units represent RAS detection of an anti-RAS antibody bound to the RBD. RAS-GTP H2122>>Mewo>A375. (D) Transfection of mutant KRASG12D (but not KRASWT) in A375 (B-RAFV600E) cells, allows the cells to induce B-RAF:C-RAF heterodimers and C-RAF kinase activation in the presence of the RAF inhibitor GDC-0879 (dosed at 0.1, 1, 10 mM). C-RAF was immunoprecipitated from control and inhibitor-treated cells and assayed for protein activity and B-RAF heterodimerization. Total C-RAF levels shown by WB in the immunoprecipitate indicate loading for each lane.

FIGS. 21A, B, C and D depict measurements of basal and EGF-stimulated pERK knockdown by Raf inhibitors in B-RafV600E and WT B-Raf cell lines. (A) Table of genotype and EGFR levels among lines tested. (B) Measurement of basal and stimulated pERK levels: cells were treated with 0.0004-10 mM compound in serum free media for 1 hour. For stimulation 20 ng/ml EGF was added for 5 min before cells were lysed. Lysates were transferred to an MSD plate where phospho- and total ERK levels were measured. (C) pERK IC50 data are plotted for the two Raf inhibitors (CHR-265, 1-methyl-5-[[2-[5-(trifluoromethyl)-1H-imidazol-2-yl]-4-pyridinyl]oxy]-N-[4-(trifluoromethyl)phenyl]-1H-Benzimidazol-2-amine and GDC-0879) under basal and EGF-stimulated conditions. (D) Dose response curves of pERK induction upon 1-hr treatment of indicated WT B-Raf lines with Raf inhibitors.

FIGS. 22A and B depict EGF stimulation rendering phospho-MEK levels and cellular proliferation of B-RAF V600E mutant cell lines resistant to RAF inhibitor. (A) Cells were treated with 0.0004-10 mM compound in serum free media for 1 hour. For stimulation 20 ng/ml EGF was added for 5 min before cells were lysed. Lysates were transferred to an MSD plate where phospho- and total MEK levels were measured. Phospho-MEK (pMEK) 1050 data are plotted for the two Raf inhibitors indicated under basal and EGF-stimulated conditions. GDC-0879 is more effective in knocking down phospho-MEK levels because it has a lower adjusted 1050 against wildtype C-RAF and B-RAF isoforms than PLX4720. (B) EGF treatment renders B-RAFV600E cells resistant to RAF inhibitors but combination with Tarceva (or MEK inhibitor, e.g. PD-0325901) overcomes that resistance. Cells were dosed with indicated inhibitors, either alone or in combination in the presence of 20 ng/ml EGF in the media.

FIG. 23 depicts EGF stimulation inducing B-RAF and C-RAF activity in B-RAFV600E mutant lines (LOX, 888 are melanoma while HT29 is colon). All cell lines express surface EGFR levels. 888 is homozygous for the B-RAF V600E allele, all others are lines are heterozygous, therefore carry a wildtype B-RAF allele as well. The heterozygous cell lines induce both B-RAF and C-RAF activity, while the homozygous line induces only C-RAF activity. This wildtype RAF activity can not be inhibited by B-RAF V600E selective RAF inhibitors, therefore the phospho-MEK induced levels by EGF are resistant to RAF inhibition in these lines, while endogenous phospho-MEK levels driven by B-RAF V600E are sensitive to B-RAF V600E selective RAF inhibitors.

FIG. 24 depicts a trend towards a negative correlation between high EGF mRNA levels (x axis) and RAF inhibitor 1050 (uM, in y axis). Cellular efficacy data are shown for B-RAF V600E melanoma cell lines and represent RAF inhibitors that are biochemically selective for the B-RAF V600E isoform with lower respective biochemical and cellular potencies against wildtype RAF isoforms.

FIG. 25 depicts RAS-GTP levels in various tumor types. RAS-GTP levels are low in K-RAS^{WT} tumors, and high in tumors bearing mutated K-RAS, for example, H2122 tumors. Ras-GTP levels were determined by RBD-Elisa assay.

FIG. 26 depicts Ras-GTP levels in B-Raf V600E cells with (+EGF) and without (NI) induction of EGF. EGF stimulation increases Ras-GTP levels in BRAF V600E cells.

FIG. 27 depicts pERK levels in B-Raf V600E cells with (stim) and without (unstim) induction of EGF. EGF stimulation increases Ras-GTP levels in BRAF V600E cells leading to an increase in pERK levels in B-RAF V600E cell lines through activation of C-Raf (see C-Raf activation shown in FIG. 23). All 4 cell lines are B-Raf V600E mutant, but among those, A375 has the lowest Ras-GTP levels (lowest levels of active Ras) and does not show robust induction of pMEK and pERK levels in response to EGF. A375 cells are known to be sensitive to Raf inhibitors.

FIG. 28 depicts pMEK levels in B-Raf V600E cells with (stim) and without (unstim) induction of EGF. EGF stimulation increases Ras-GTP levels in BRAF V600E cells leading to an increase in pMEK levels in B-RAF V600E cell lines through activation of C-Raf (see C-Raf activation shown in FIG. 23).

FIG. 29 summarizes certain RAF inhibitor (GDC-0879, PLX-4720 and “Raf inh a” which is 2,6-difluoro-N-(3-methoxy-1H-pyrazolo[3,4-b]pyridin-5-yl)-3-(propylsulfonamido)benzamide) potencies for blocking cellular pERK induction in response to EGF stimulation. BRAF V600E cells expressing EGFR were serum starved and then either left unstimulated (−EGF) or stimulated with EGF (+EGF) in the presence of the indicated RAF inhibitors at different doses. pERK inhibition curves were generated and IC₅₀ values graphed. GDC-0879, as shown in FIG. 1, can more efficiently block wildtype RAF signaling while the remaining two inhibitors are BRAF V600E selective.

FIG. 30 depicts how HGF stimulation (+HGF) leads to pERK induction in cells overexpressing c-MET. This induction is not blocked by RAF inhibitors. However, basal pERK levels that are driven by BRAF V600E are effectively blocked by RAF inhibitors. This demonstrates that c-MET signaling is also through wildtype RAF isoforms.

Therefore, aberrant expression of receptor tyrosine kinase (RTKs), including EGFR, or aberrant induction by the corresponding ligands, can render cells resistant to RAF inhibitors.

FIG. 31 shows how EGFR expression is associated with resistance to RAF inhibitors among B-RAFV600E cells. This graph represent cellular viability EC50 values (uM) of B-RAF V600E mutant melanoma and colon cell lines that were treated with a RAF inhibitor for 4 days before viability determination. EGFR levels were determined by western blot and classified as negative when no band could be detected by western blot with an anti-EGFR antibody of cell lysates. Among EGFR positive cell lines, there is a range of expression from low to moderate and high. The single EGFR negative cell line that is resistant (>20 uM EC50) is PTEN null.

FIGS. 32A-C depict combination studies of RAF inhibitor and EGFR inhibitor (Tarceva) in colon tumor lines with different levels of EGFR expression.

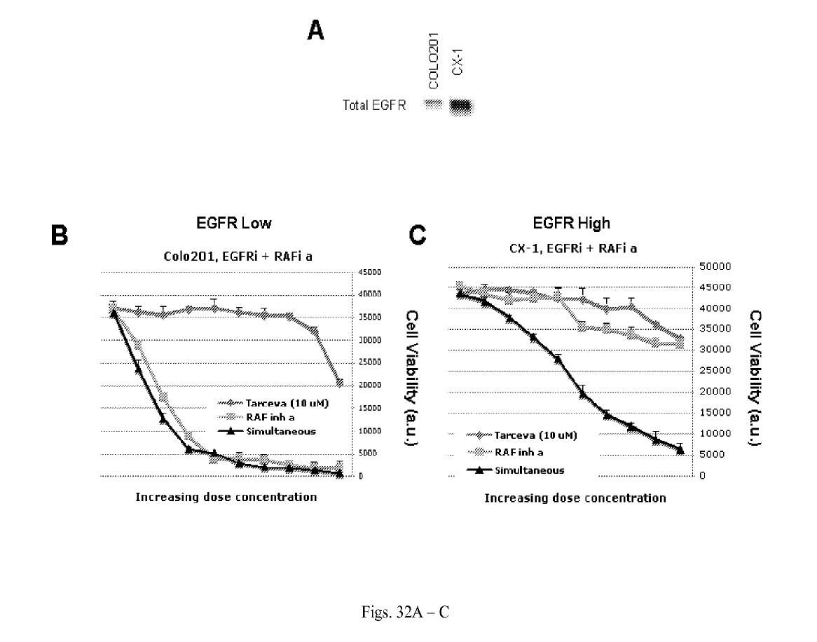

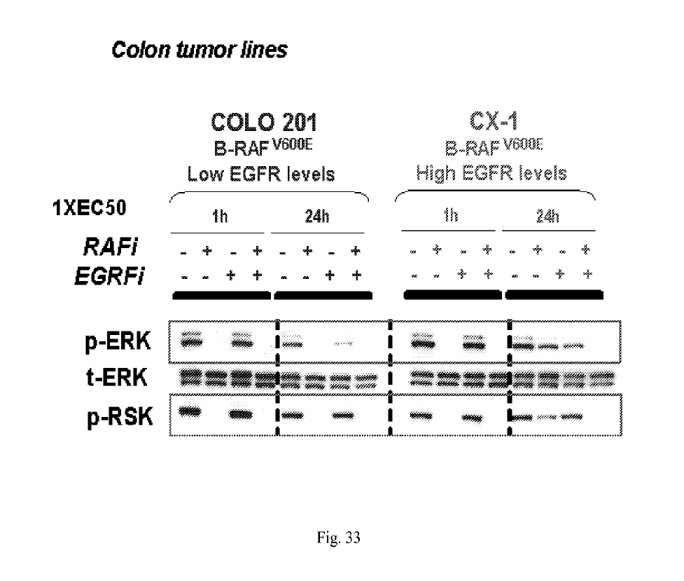

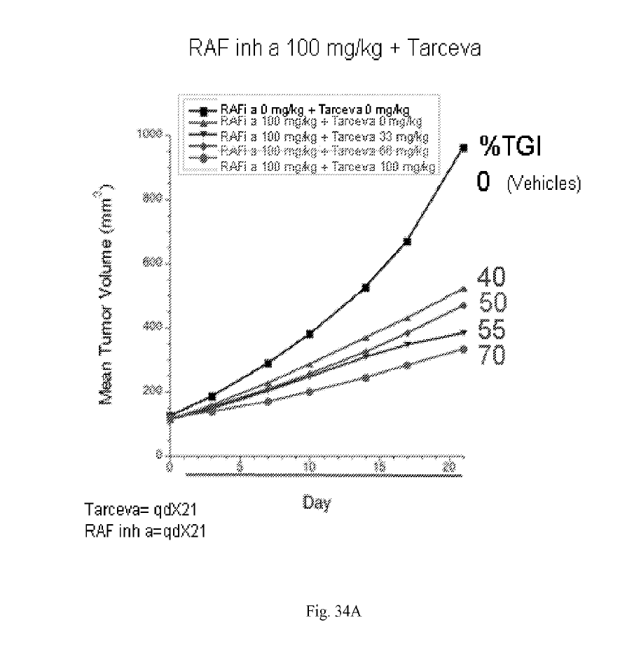

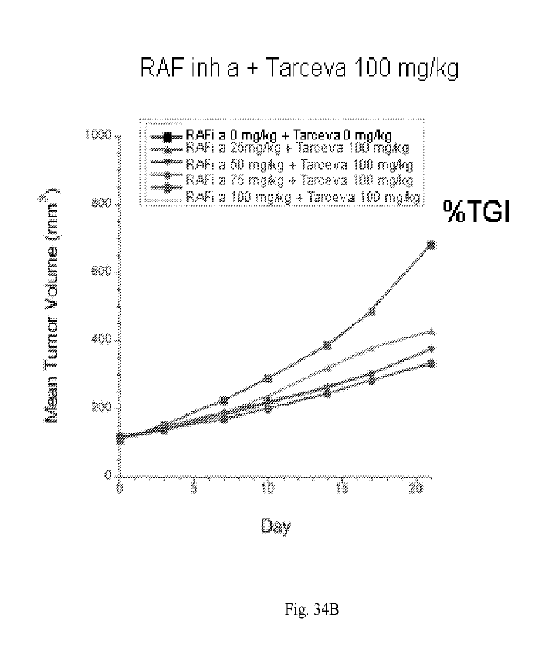

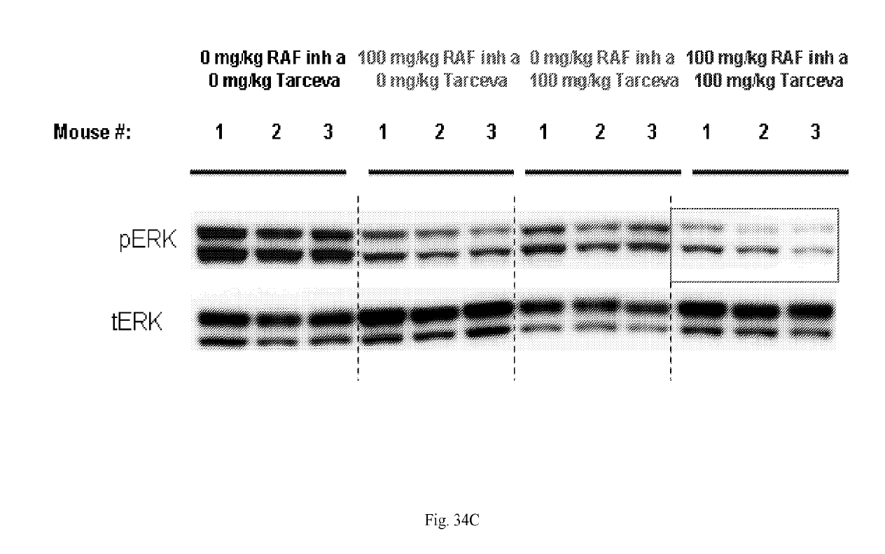

In FIG. 32A, western blot of lysates from two BRAF V600E colon lines shows their different levels of total EGFR: COLO201 has low EGFR levels while CX-1 has relatively high EGFR levels.

In FIG. 32B, the effects of combination treatment of COLO201 cells with either RAF inhibitor alone, Tarceva alone or combination of RAF inhibitor and Tarceva are shown.

In FIG. 32C, the effects of combination treatment of CX-1 cells with either RAF inhibitor alone, Tarceva alone or combination of RAF inhibitor and Tarceva are shown. Neither RAF inhibitor alone nor Tarceva alone suppress proliferation as effectively as the combination. Both inhibitors show good synergy when administered together to CX-1 cells.

Therefore, among EGFR expressing BRAFV600E cells, high levels of EGFR predict strong synergy between RAF inhibitors and EGFR inhibitors. Particularly in colon cancer, where high EGFR expression is prevalent among BRAFV600E tumors, combination of these RAF inhibitors and Tarceva show synergy in inhibiting proliferation of tumor cells.

FIG. 33 shows a mechanistic basis for synergy between RAF inhibitors and Tarceva in BRAFV600E tumor cells expressing high EGFR levels. Western blot were prepared of cells treated for either 1 hour or 24 hours with either no inhibitors (lanes 1, 5, 9, 13) or with RAF inhibitor alone (lanes 2, 6, 10, 14), Tarceva alone (lanes 3, 7, 11, 15) or combination of RAF inhibitor and Tarceva (lanes 4, 8, 12, 16) at a concentration equal to their cellular EC50 value. The 24 hour timepoint shows that ERK phosphorylation in B-RAFV600E mutant cells with high EGFR expression (CX-1) has reduced sensitive to inhibition by RAF inhibitors and requires RAF inhibitor and EGFR inhibitor combination for maximal efficacy. A portion of the activation signal to ERK comes from wildtype RAF that is activated downstream of EGFR and may not be blocked by the BRAF V600E selective RAF inhibitor.

FIGS. 34A-C show results from the interaction and efficacy of the RAF inhibitor a and Erlotinib (Tarceva) given in combination to NCR nude (Taconic) mice bearing subcutaneous HT-29 BRAF V600E human colorectal carcinoma xenografts. In FIG. 34A, RAF inh a was given at 100 mg/kg with increasing doses of Tarceva. In FIG. 34B, Tarceva was given to all animals with increasing concentrations of RAF inh a. Increased efficacy was observed when both compounds were administered in combination. In FIG. 34C, lysates from tumors treated with the indicated doses of inhibitors in FIGS. 34A and B were analyzed for phospho-ERK (pERK) levels by western blot. The RAF inhibitor a and Tarceva synergized in decreasing phospho-ERK levels in the tumors when co-administered in mice.

### DETAILED DESCRIPTION OF THE INVENTION

In one embodiment, the subject matter disclosed herein relates to a method of identifying a patient nonresponsive to treatment with a B-Raf inhibitor, comprising determining the amount of expression or induction of RTKs and/or their ligands. The methods involve determining the expression or induction levels of certain RTKs and/or their ligands in a sample, whereby overexpression of RTKs and/or their ligands correlate non-responsiveness to B-Raf inhibitor treatment. In an embodiment, the sample expresses the B-Raf V600E mutant. Examples of RTKs that correlate to the responsiveness of B-Raf treatment include, but are not limited to EGFR and cMet. The methods also involve determining the levels of expression of certain ligands of RTKs in a sample whereby abnormally high levels of ligand expression correlates with non-responsiveness to B-Raf inhibitor treatment. Examples of ligands that correlate to the responsiveness of B-Raf treatment include, but are not limited to EGF and HGF.

In one embodiment, the subject matter disclosed herein relates to a method of identifying a patient nonresponsive to treatment with a B-Raf inhibitor, comprising determining the amount of Ras-GTP in a sample, whereby elevated amounts indicate a patient will not respond to said B-Raf inhibitor treatment. In one example, the elevated amounts are greater than amounts found in normal unstimulated samples. Methods for measuring the levels of Ras-GTP in a sample are known, for example, ELISA assays are used (e.g. Ras-GTPase ELISA assays from Upstate, Inc.). In one example, the method further comprises administering an effective amount of a MEK or ERK inhibitor to said nonresponsive patient. In another example, the method further comprises administering an effective amount of an inhibitor of EGFR signaling. In another example, the method further comprises administering an effective amount of an inhibitor of EGFR signaling in combination with a B-Raf inhibitor.

In one embodiment, the subject matter disclosed herein relates to a method of identifying a patient nonresponsive to treatment with a B-Raf inhibitor, comprising determining the level of EGF or EGFR expression in a sample, whereby overexpressed levels of either EGF or EGFR indicate a patient will not respond to said B-Raf inhibitor treatment. In one example, the amount of EGF mRNA is determined. Methods for measuring the levels of EGF and EGFR expression in a sample are known, for example, ELISA immunoassays are used (e.g. QUANTIKINE® immunoassays from R&D Systems, Inc.). In one example, the method further comprises administering an effective amount of a MEK or ERK inhibitor to said nonresponsive patient. In another example, the method further comprises administering an effective amount of an inhibitor of EGFR signaling. In another example, the method further comprises administering an effective amount of an inhibitor of EGFR signaling in combination with a B-Raf inhibitor.

In one embodiment, the subject matter disclosed herein relates to a method of identifying a patient nonresponsive to treatment with a B-Raf inhibitor, comprising determining the level of HGF or cMET expression in a sample, whereby overexpressed levels of either HGF or cMET indicate a patient will not respond to said B-Raf inhibitor treatment. In one example, the patient expresses B-Raf V600E. In one example, the amount of HGF mRNA is determined. Methods for measuring the levels of HGF and cMET expression in a sample are known, for example, quantitative RT-RealTime PCR assays are used. In another example, ELISA immunoassays are used (e.g. PhosphoDetect® cMET ELISA kits from EMD Chemicals, Inc, or the cMET Human ELISA kit from Invitrogen, Inc.). In one example, the method further comprises administering an effective amount of a cMET or HGF inhibitor to said nonresponsive patient. In another example, the method further comprises administering an effective amount of a cMET or HGF inhibitor in combination with a B-Raf inhibitor.

In one embodiment, the subject matter disclosed herein relates to a method of identifying a patient nonresponsive to treatment with a B-Raf inhibitor, comprising determining the presence or absence of a K-ras mutation, whereby the presence of a K-ras mutation indicates a patient will not respond to said B-Raf inhibitor treatment. In one example, the method further comprises administering an effective amount of a MEK or ERK inhibitor to said nonresponsive patient. In another example, the method further comprises administering an effective amount of an inhibitor of EGFR signaling. In another example, the method further comprises administering an effective amount of an inhibitor of EGFR signaling in combination with a B-Raf inhibitor.

In certain embodiments, the subject matter disclosed herein relates to a method of determining whether a tumor will respond to treatment with a B-Raf inhibitor, comprising determining in a sample of said tumor the presence of a mutant K-ras protein or gene whereby the presence of a mutant K-ras protein or gene indicates that the tumor will not respond to treatment with a B-Raf inhibitor. In one example, the method further comprises administering an effective amount of a MEK or ERK inhibitor to said nonresponsive tumor. In another example, the method further comprises administering an effective amount of an inhibitor of EGFR signaling. In another example, the method further comprises administering an effective amount of an inhibitor of EGFR signaling in combination with a B-Raf inhibitor.

In certain embodiments, a method of predicting whether a patient will be nonresponsive to treatment with a B-Raf inhibitor is provided. In certain embodiments, the method comprises determining the presence or absence of a K-ras mutation in a tumor of the patient, wherein the K-ras mutation is in codon 12 or codon 13. In certain embodiments, if a K-ras mutation is present, the patient is predicted to be nonresponsive to treatment with a B-Raf inhibitor.

In certain embodiments, a method of predicting whether a tumor will be nonresponsive to treatment with a B-Raf inhibitor is provided. In certain embodiments, the method comprises determining the presence or absence of a K-ras mutation in a sample of said tumor, wherein the K-ras mutation is in codon 12 or codon 13. In certain embodiments, the presence of the K-ras mutation indicates that the tumor will be nonresponsive to treatment with a B-Raf inhibitor.

In certain embodiments, a method of stratifying a human subject in a treatment protocol is provided. The method comprises determining the presence of a mutant K-ras gene or protein thereof in a sample from the subject whereby the presence of a mutant K-ras gene or protein indicates that the subject will not respond to B-Raf inhibitor treatment, and excluding the subject from treatment with a B-Raf inhibitor. This method can include stratifying the subject to a particular subgroup in, for example, a clinical trial. In another embodiment, the method further comprises administering an effective amount of a MEK or ERK inhibitor to said subject having said mutant K-ras gene or protein. In another example, the method further comprises administering an effective amount of an inhibitor of EGFR signaling. In another example, the method further comprises administering an effective amount of an inhibitor of EGFR signaling in combination with a B-Raf inhibitor.

In an embodiment, a method of classifying a breast, lung, colon, ovarian, thyroid, melanoma or pancreatic tumor is provided. The method comprises the steps of: obtaining or providing a tumor sample; detecting expression or activity of a (i) a gene encoding the B-Raf V600E mutant and (ii) a gene encoding a k-Ras mutant in the sample. The method can further comprise classifying the tumor as belonging to a tumor subclass based on the results of the detecting step; and selecting a treatment based on the classifying step, wherein said treatment is other than a B-Raf V600E specific inhibitor if said k-RAS mutant is overexpressed in said tumor sample. In one example, the treatment comprises administering an effective amount of a MEK or ERK inhibitor to said nonresponsive tumor. In another example, the method further comprises administering an effective amount of an inhibitor of EGFR signaling. In another example, the method further comprises administering an effective amount of an inhibitor of EGFR signaling in combination with a B-Raf inhibitor.

In another embodiment, a method of treating colorectal or lung cancer is provided. The method comprises determining whether the cancer is K-ras or B-Raf driven, whereby in treating such a cancer that is determined to be K-ras driven, the treatment does not include a B-Raf inhibitor. In one example, the treatment comprises administering an effective amount of a MEK or ERK inhibitor to said K-ras driven cancer. Also provided is a kit comprising specific material for detecting whether the cancer is K-ras driven or B-Raf driven and instructions for identifying a patient or tumor that is non-responsive to B-Raf inhibitor treatment.

In certain embodiments, determining the presence or absence of one or more K-ras mutations in a subject comprises determining the presence or amount of expression of a mutant K-ras polypeptide in a sample from the subject. In certain embodiments, determining the presence or absence of one or more K-ras mutations in a subject comprises determining the presence or amount of transcription or translation of a mutant K-ras polynucleotide in a sample from the subject.

In certain embodiments, determining the presence or absence of one or more K-ras mutations in a subject comprises determining the presence or amount of expression of a polypeptide comprising at least one amino acid sequence selected from the group consisting of the following SEQ ID NOs. listed in US2009/0075267: SEQ ID NO: 4, SEQ ID NO: 6, SEQ ID NO: 8, SEQ ID NO: 10, SEQ ID NO: 12, SEQ ID NO: 14, and SEQ ID NO: 16. In certain embodiments, determining the presence or absence of one or more K-ras mutations in a subject comprises determining the presence or amount of transcription or translation of a polynucleotide encoding at least one amino acid sequence selected from the group consisting of the following SEQ ID NOs. listed in US2009/0075267: SEQ ID NO: 4, SEQ ID NO: 6, SEQ ID NO: 8, SEQ ID NO: 10, SEQ ID NO: 12, SEQ ID NO: 14, and SEQ ID NO: 16 in a sample from the subject.

In certain embodiments, determining the presence or absence of a polynucleotide encoding a mutant K-ras polypeptide is provided. In certain embodiments, a method of determining the presence or absence of a polynucleotide encoding a mutant K-ras polypeptide in a sample comprises (a) exposing a sample to a probe which hybridizes to a polynucleotide encoding a region of a mutant K-ras polypeptide, wherein the region comprises at least one K-ras mutation selected from G12S, G12V, G12D, G12A, G12C, G13A, and G13D, and (b) determining the presence or absence of a polynucleotide encoding a mutant K-ras polypeptide in the sample. In certain embodiments, a method of determining the presence or absence of a mutant K-ras polypeptide in a sample comprises (a) exposing a sample to a probe which hybridizes to a polynucleotide encoding a region of a mutant K-ras polypeptide, wherein the region comprises at least one K-ras mutation selected from G12S, G12V, G12D, G12A, G12C, G13A, and G13D, and (b) determining the presence or absence of a mutant K-ras polypeptide in the sample.

In certain embodiments, determining the presence or absence of a polynucleotide encoding a mutant B-Raf polypeptide is provided. In certain embodiments, a method of determining the presence or absence of a polynucleotide encoding a mutant B-Raf polypeptide in a sample comprises (a) exposing a sample to a probe which hybridizes to a polynucleotide encoding a region of a mutant B-Raf polypeptide, wherein the region comprises a V600E mutation, and (b) determining the presence or absence of a polynucleotide encoding a mutant B-Raf polypeptide in the sample. In certain embodiments, a method of determining the presence or absence of a mutant B-Raf polypeptide in a sample comprises (a) exposing a sample to a probe which hybridizes to a polynucleotide encoding a region of a mutant B-raf polypeptide, wherein the region comprises a V600E mutation, and (b) determining the presence or absence of a mutant B-Raf polypeptide in the sample.

In certain embodiments, a kit for detecting a polynucleotide encoding a mutant K-ras polypeptide in a subject is provided. In certain such embodiments, the kit comprises a probe which hybridizes to a polynucleotide encoding a region of a mutant K-ras polypeptide, wherein the region comprises at least one K-ras mutation selected from G12S, G12V, G12D, G12A, G12C, G13A, and G13D. In certain embodiments, the kit further comprises two or more amplification primers. In certain embodiments, the kit further comprises a detection component. In certain embodiments, the kit further comprises a nucleic acid sampling component. The kit can optionally contain material for detecting a B-Raf mutation. These materials are known in the art. The combination of a kit capable of detecting K-ras and B-Raf mutant genes or proteins is particularly useful in treating colon and lung cancer. Included in the kit are instructions for identifying a patient or tumor that is non-responsive to B-Raf inhibition when the cancer is K-ras driven. RAS-driven cancers are known in the art. A Ras-driven cancer is any cancer or tumor in which abherant activity of a Ras protein results in production of a transformed cell or the formation of cancer or a tumor.

In certain embodiments, for those samples, tumors, cancers, subjects or patients determined to be unresponsive to a B-Raf inhibitor, the methods further comprise administering an effective amount of a MEK inhibitor to said unresponsive samples, tumors, cancers, subjects or patients.

In certain embodiments, for those samples, tumors, cancers, subjects or patients determined to be unresponsive to a B-Raf inhibitor, the methods further comprise administering an effective amount of a ERK inhibitor to said unresponsive samples, tumors, cancers, subjects or patients. In another example, the method further comprises administering an effective amount of an inhibitor of EGFR signaling. In another example, the method further comprises administering an effective amount of an inhibitor of EGFR signaling in combination with a B-Raf inhibitor.

EGFR signaling can be inhibited by a variety of methods, including inhibiting EGFR kinase activity, binding to the extracellular domain of EGFR to inhibit activation or by inhibiting the activity and signaling of EGF ligand.

Inhibitors of EGFR signaling are known in the art and include, for example, erlotinib (TARCEVA®), gefitinib (IRESSA®), lapatinib, pelitinib, Cetuximab, panitumumab, zalutumumab, nimotuzumab and matuzumab, and those described in U.S. Pat. No. 5,747,498.

B-Raf inhibitors are known in the art and include, for example, sorafenib, PLX4720, PLX-3603, GSK2118436, GDC-0879, N-(3-(5-(4-chlorophenyl)-1H-pyrrolo[2,3-b]pyridine-3-carbonyl)-2,4-difluorophenyl)propane-1-sulfonamide, and those described in WO2007/002325, WO2007/002433, WO2009111278, WO2009111279, WO2009111277, WO2009111280 and U.S. Pat. No. 7,491,829.

cMET inhibitors are known in the art, and include, but are not limited to, AMG208, ARQ197, ARQ209, PHA665752 (3Z)-5-[(2,6-dichlorobenzyl)sulfonyl]-3-[(3,5-dimethyl-4-{[(2R)-2-(pyrrolidin-1-ylmethyl)pyrrolidin-1-yl]carbonyl}-1H-pyrrol-2-yl)methylene]-1,3-dihydro-2H-indol-2-one, N-(4-(3-((3S,4R)-1-ethyl-3-fluoropiperidin-4-ylamino)-1H-pyrazolo[3,4-b]pyridin-4-yloxy)-3-fluorophenyl)-2-(4-fluorophenyl)-3-oxo-2,3-dihydropyridazine-4-carboxamide and SU11274, and those described in U.S. Pat. No. 7,723,330.

MEK inhibitors are known in the art, and include, but are not limited to, ARRY-162, AZD8330, AZD6244, U0126, GDC-0973, PD184161 and PD98059, and those described in WO2003047582, WO2003047583, WO2003047585, WO2003053960, WO2007071951, WO2003077855, WO2003077914, WO2005023251, WO2005051300, WO2005051302, WO2007022529, WO2006061712, WO2005028426, WO2006018188, US20070197617, WO2008101840, WO2009021887, WO2009153554, US20090275606, WO2009129938, WO2009093008, WO2009018233, WO2009013462, WO2008125820, WO2008124085, WO2007044515, WO2008021389, WO2008076415 and WO2008124085.

ERK inhibitors are known in the art, and include, but are not limited to, FR180204 and 3-(2-Aminoethyl)-5-(4-ethoxyphenyl)methylene)-2,4-thiazolidinedione, and those described in WO2006071644, WO2007070398, WO2007097937, WO2008153858, WO2008153858, WO2009105500 and WO2010000978.

Any known method for detecting a mutant K-ras gene or protein is suitable for the method disclosed herein. Particular mutations detected in exon 1 are: G12C; G12A; G12D; G12R; G12S; G12V; G13C; G13D. Methods for determining the presence of K-ras mutations are also analogous to those used to identify K-ras and EGFR mutations, for example the K-ras oligos for PCR listed as SEQ ID Nos. 55, 56, 57 and 58 as described in published U.S. Patent App. No. US2009/0202989A1, herein incorporated by reference in its entirety. By way of example, other methods for detecting a mutant K-ras gene or protein, and the primers, oligos and SEQ ID Nos. are disclosed in published U.S. Patent Application Nos. US2009/0202989A1, US2009/0075267A1, US20090143320, US20040063120 and US2007/0003936. The techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference.

Certain methods of detecting a mutation in a polynucleotide are known in the art. Certain exemplary methods include, but are not limited to, sequencing, primer extension reactions, electrophoresis, picogreen assays, oligonucleotide ligation assays, hybridization assays, TaqMan assays, SNPlex assays, and assays described, e.g., in U.S. Pat. Nos. 5,470,705, 5,514,543, 5,580,732, 5,624,800, 5,807,682, 6,759,202, 6,756,204, 6,734,296, 6,395,486, and U.S. Patent Publication No. US 2003-0190646 A1.

In certain embodiments, detecting a mutation in a polynucleotide comprises first amplifying a polynucleotide that may comprise the mutation. Certain methods for amplifying a polynucleotide are known in the art. Such amplification products may be used in any of the methods described herein, or known in the art, for detecting a mutation in a polynucleotide.

Certain methods of detecting a mutation in a polypeptide are known in the art. Certain exemplary such methods include, but are not limited to, detecting using a specific binding agent specific for the mutant polypeptide. Other methods of detecting a mutant polypeptide include, but are not limited to, electrophoresis and peptide sequencing.

Certain exemplary methods of detecting a mutation in a polynucleotide and/or a polypeptide are described, e.g., in Schimanski et al. (1999) Cancer Res., 59: 5169-5175; Nagasaka et al. (2004) J. Clin. Oncol., 22: 4584-4596; PCT Publication No. WO 2007/001868 A1; U.S. Patent Publication No. 2005/0272083 A1; and Lievre et al. (2006) Cancer Res. 66: 3992-3994.

In certain embodiments, microarrays comprising one or more polynucleotides encoding one or more mutant K-ras polypeptides are provided. In certain embodiments, microarrays comprising one or more polynucleotides complementary to one or more polynucleotides encoding one or more mutant K-ras polypeptides are provided. In certain embodiments, microarrays comprising one or more polynucleotides encoding one or more mutant B-Raf polypeptides are provided. In certain embodiments, microarrays comprising one or more polynucleotides complementary to one or more polynucleotides encoding one or more mutant B-Raf polypeptides are provided.

In certain embodiments, the presence or absence of one or more mutant K-ras polynucleotides in two or more cell or tissue samples is assessed using microarray technology. In certain embodiments, the quantity of one or more mutant K-ras polynucleotides in two or more cell or tissue samples is assessed using microarray technology.

In certain embodiments, the presence or absence of one or more mutant B-Raf polynucleotides in two or more cell or tissue samples is assessed using microarray technology. In certain embodiments, the quantity of one or more mutant B-Raf polynucleotides in two or more cell or tissue samples is assessed using microarray technology.

In certain embodiments, the presence or absence of one or more mutant K-ras polypeptides in two or more cell or tissue samples is assessed using microarray technology. In certain such embodiments, mRNA is first extracted from a cell or tissue sample and is subsequently converted to cDNA, which is hybridized to the microarray. In certain such embodiments, the presence or absence of cDNA that is specifically bound to the microarray is indicative of the presence or absence of the mutant K-ras polypeptide. In certain such embodiments, the expression level of the one or more mutant K-ms polypeptides is assessed by quantitating the amount of cDNA that is specifically bound to the microarray.

In certain embodiments, the presence or absence of one or more mutant B-raf polypeptides in two or more cell or tissue samples is assessed using microarray technology. In certain such embodiments, mRNA is first extracted from a cell or tissue sample and is subsequently converted to cDNA, which is hybridized to the microarray. In certain such embodiments, the presence or absence of cDNA that is specifically bound to the microarray is indicative of the presence or absence of the mutant B-Raf polypeptide. In certain such embodiments, the expression level of the one or more mutant B-Raf polypeptides is assessed by quantitating the amount of cDNA that is specifically bound to the microarray.

In certain embodiments, microarrays comprising one or more specific binding agents to one or more mutant K-ras polypeptides are provided. In certain such embodiments, the presence or absence of one or more mutant K-ras polypeptides in a cell or tissue is assessed. In certain such embodiments, the quantity of one or more mutant K-ras polypeptides in a cell or tissue is assessed.

In certain embodiments, microarrays comprising one or more specific binding agents to one or more mutant B-Raf polypeptides are provided. In certain such embodiments, the presence or absence of one or more mutant B-Raf polypeptides in a cell or tissue is assessed. In certain such embodiments, the quantity of one or more mutant B-raf polypeptides in a cell or tissue is assessed.

All references cited herein, including patents, patent applications, papers, textbooks, and the like, and the references cited therein, to the extent that they are not already, are hereby incorporated herein by reference in their entirety. In the event that one or more of the documents incorporated by reference defines a term that contradicts that term's definition in this application, this application controls. The section headings used herein are for organizational purposes only and are not to be construed as limiting the subject matter described.

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

The term “B-Raf inhibitor” refers to any compound or agent that inhibits decreases the activity of a B-Raf kinase. Such an inhibitor may also inhibit other kinases, including other raf kinases. A “specific B-Raf kinase inhibitor” refers to an inhibitor that has selectivity for a mutant B-Raf, such as a mutation at the valine residue at amino acid position 600, e.g., a V600E mutation, compared to the wild-type B-Raf. Such an inhibitor is at least two times, more often at least three times or more, as potent compared to the wild-type B-Raf. The potency can also be compared in terms of IC₅₀ values for cellular assays that measure growth inhibition.

The term “treatment protocol” refers to a therapeutic regimen or course of administering one or more agents to treat a disorder or disease. This includes clinical trials.

The terminology “X#Y” in the context of a mutation in a polypeptide sequence is art-recognized, where “#” indicates the location of the mutation in terms of the amino acid number of the polypeptide, “X” indicates the amino acid found at that position in the wild-type amino acid sequence, and “Y” indicates the mutant amino acid at that position. For example, the notation “G12S” with reference to the K-ras polypeptide indicates that there is a glycine at amino acid number 12 of the wild-type K-ras sequence, and that glycine is replaced with a serine in the mutant K-ras sequence.

The terms “mutant K-ras polypeptide” and “mutant K-ras protein” are used interchangeably, and refer to a K-ras polypeptide comprising at least one K-ras mutation selected from G12S, G12V, G12D, G12A, G12C, G13A, and G13D. Certain exemplary mutant K-ras polypeptides include, but are not limited to, allelic variants, splice variants, derivative variants, substitution variants, deletion variants, and/or insertion variants, fusion polypeptides, orthologs, and interspecies homologs. In certain embodiments, a mutant K-ras polypeptide includes additional residues at the C- or N-terminus, such as, but not limited to, leader sequence residues, targeting residues, amino terminal methionine residues, lysine residues, tag residues and/or fusion protein residues.

The terms “mutant B-Raf polypeptide” and “mutant B-Raf protein” are used interchangeably, and refer to a B-Raf polypeptide comprising V600E mutation. Certain exemplary mutant B-Raf polypeptides include, but are not limited to, allelic variants, splice variants, derivative variants, substitution variants, deletion variants, and/or insertion variants, fusion polypeptides, orthologs, and interspecies homologs. In certain embodiments, a mutant B-Raf polypeptide includes additional residues at the C- or N-terminus, such as, but not limited to, leader sequence residues, targeting residues, amino terminal methionine residues, lysine residues, tag residues and/or fusion protein residues.

The terms “mutant K-ras polynucleotide”, “mutant K-ras oligonucleotide,” and “mutant K-ras nucleic acid” are used interchangeably, and refer to a polynucleotide encoding a K-ras polypeptide comprising at least one K-ras mutation selected from G12S, G12V, G12D, G12A, G12C, G13A, and G13D.

The terms “mutant B-Raf polynucleotide”, “mutant B-Raf oligonucleotide,” and “mutant B-Raf nucleic acid” are used interchangeably, and refer to a polynucleotide encoding a B-Raf polypeptide comprising a V600E mutation.

The term “agent” is used herein to denote a chemical compound, a mixture of chemical compounds, a biological macromolecule, or an extract made from biological materials.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term patient includes human and animal subjects.

The terms “mammal” and “animal” for purposes of treatment refers to any animal classified as a mammal, including humans, domestic and farm animals, and zoo, sports, or pet animals, such as dogs, horses, cats, cows, etc. Preferably, the mammal is human.

The term “disease state” refers to a physiological state of a cell or of a whole mammal in which an interruption, cessation, or disorder of cellular or body functions, systems, or organs has occurred.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “responsive” as used herein means that a patient or tumor shows a complete response or a partial response after administering an agent, according to RECIST (Response Evaluation Criteria in Solid Tumors). The term “nonresponsive” as used herein means that a patient or tumor shows stable disease or progressive disease after administering an agent, according to RECIST. RECIST is described, e.g., in Therasse et al., February 2000, “New Guidelines to Evaluate the Response to Treatment in Solid Tumors,” J. Natl. Cancer Inst. 92(3): 205-216, which is incorporated by reference herein in its entirety.

A “disorder” is any condition that would benefit from one or more treatments. This includes chronic and acute disorders or disease including those pathological conditions which predispose the mammal to the disorder in question. Non-limiting examples of disorders to be treated herein include benign and malignant tumors, leukemias, and lymphoid malignancies. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In particular, the present method is suitable for breast, colorectal, ovarian, pancreatic or lung cancer. More particularly, the cancer is colon, lung or ovarian. The cancer may be a Ras-driven cancer.

A disease or condition related to a mutant K-ras includes one or more of the following: a disease or condition caused by a mutant K-ras gene or protein; a disease or condition contributed to by a mutant K-ras gene or protein; and a disease or condition that is associated with the presence of a mutant K-ras gene or protein. In certain embodiments, a disease or condition related to a mutant K-ras is a cancer.

A “disease or condition related to a mutant K-ras polypeptide” includes one or more of the following: a disease or condition caused by a mutant K-ras polypeptide; a disease or condition contributed to by a mutant K-ras polypeptide; a disease or condition that causes a mutant K-ras polypeptide; and a disease or condition that is associated with the presence of a mutant K-ras polypeptide. In certain embodiments, the disease or condition related to a mutant K-ras polypeptide may exist in the absence of the mutant K-ras polypeptide. In certain embodiments, the disease or condition related to a mutant K-ras polypeptide may be exacerbated by the presence of a mutant K-ras polypeptide. In certain embodiments, a disease or condition related to a mutant K-ras polypeptide is a cancer.

The following examples, including the experiments conducted and results achieved are provided for illustrative purpose only and are not to be construed as limiting upon the claims.

### EXAMPLES

### Example 1

### B-RAF Deletion and Pharmacological Inhibition Enhances K-Ras Driven Tumorigenesis

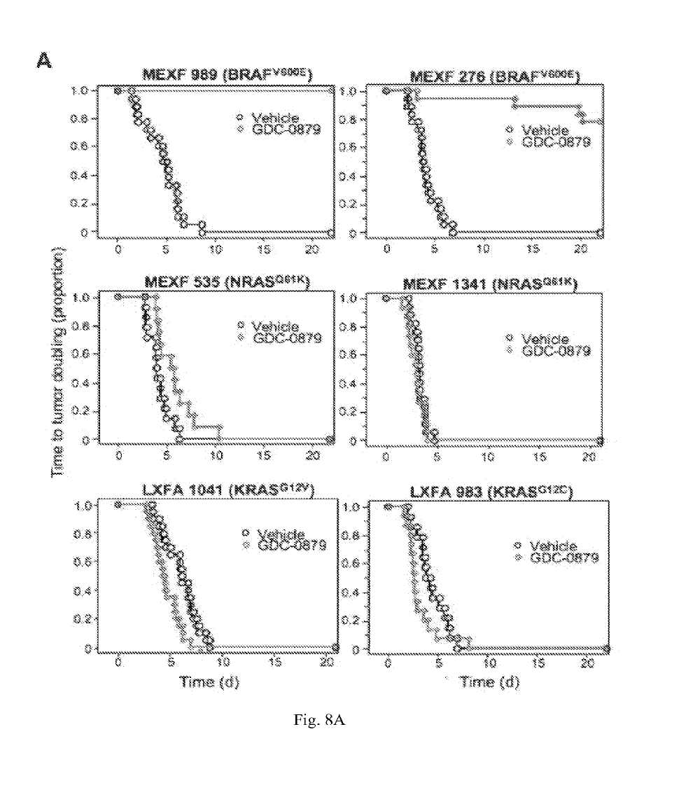

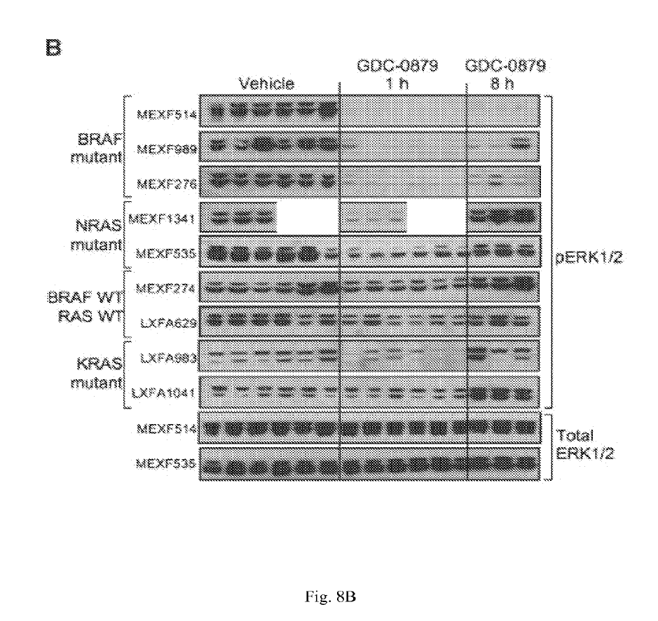

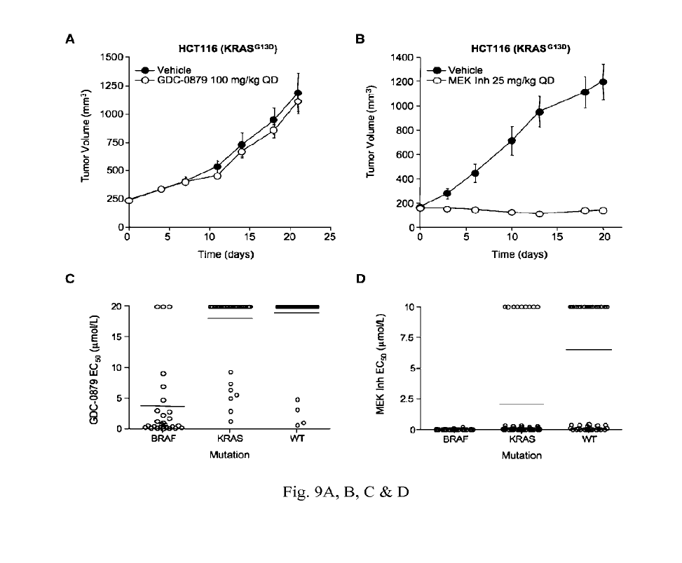

The Ras GTPase family controls numerous downstream signaling cascades in response to signals that regulate cellular processes including proliferation and survival. While Ras is one of the most prevalent targets for gain-of-function mutations in human tumors, questions remain regarding how the Ras effector pathway functions in mutant K-ras-driven tumorigenesis. Since an important function of K-ras involves B-Raf activation within the canonical MAPK signaling pathway, we initiated a study to determine B-Raf's role in the context of mutant K-ras-driven tumor promotion and maintenance. In some K-ras mutant tumors, B-Raf inhibition not only failed to show any tumor benefit, it even accelerated tumor growth. See, FIG. 8A, showing time to tumor doubling.

Adenovirus expressing the Cre recombinase was delivered to the lungs of genetically engineered mice possessing a conditional K-ras^{G12D} allele (K-ras^{LSL-G12D}) and either 0, 1 or both copies of the B-raf gene flanked by LoxP sites (B-raf^{CKO}). This procedure results in expression of mutant K-ras^{G12D} in the presence or absence of one or both B-raf alleles deleted within the mouse lung. Surprisingly, B-Raf deletion significantly enhances lung tumor number and burden and decreases overall survival. When a highly specific small-molecule inhibitor that targets B-Raf in a murine non-small cell lung carcinoma line harboring the K-ras^{G12D} mutation was used, we observed an increase in cell proliferation and soft agar colony formation. Further investigation revealed that treating K-ras^{G12D} expressing cells with the B-Raf inhibitor enhanced MEK and Erk phosphorylation. Therefore, these data suggests that while B-Raf deletion does not inhibit K-ras-driven tumor initiation and disease progression, its presence may play a pivotal role in establishing negative feedback regulation of constitutive mutant K-ras activity.

### Example 2

### Understanding RAF Signaling in B-RAF^{V600E} Mutant Versus Wild-Type Tumors

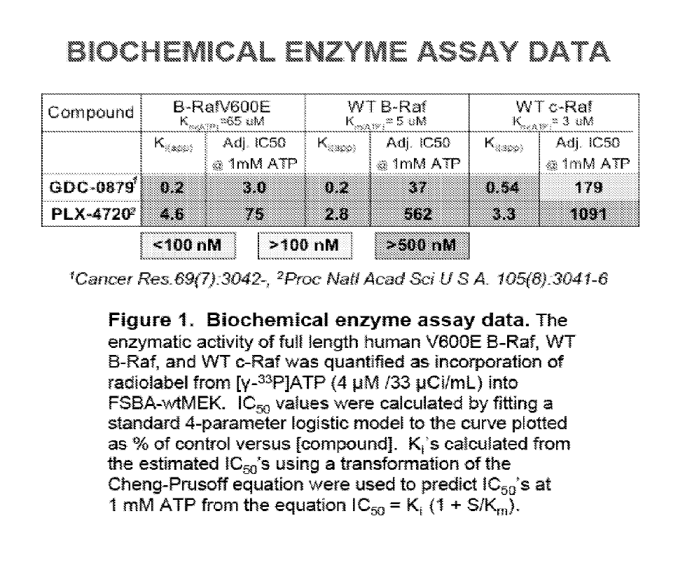

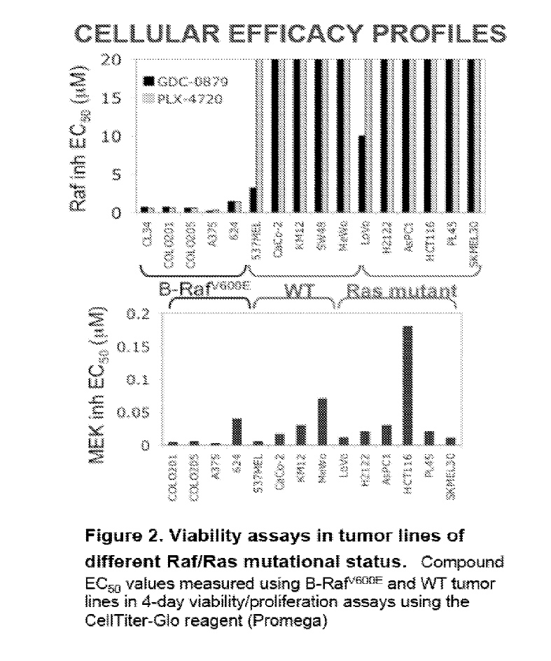

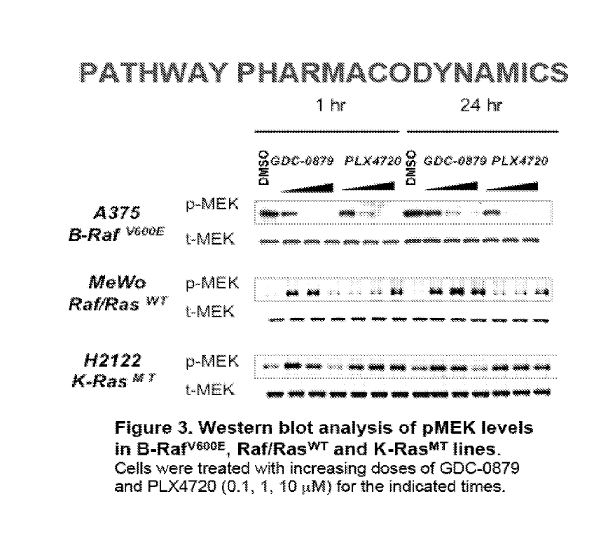

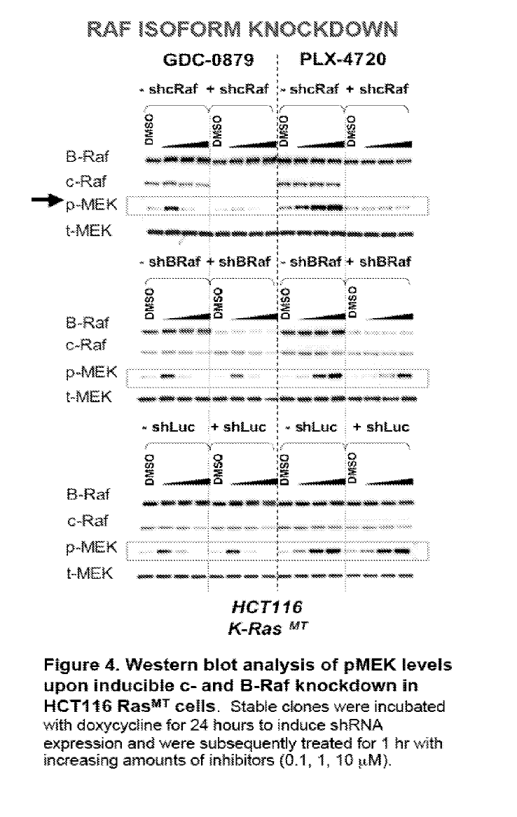

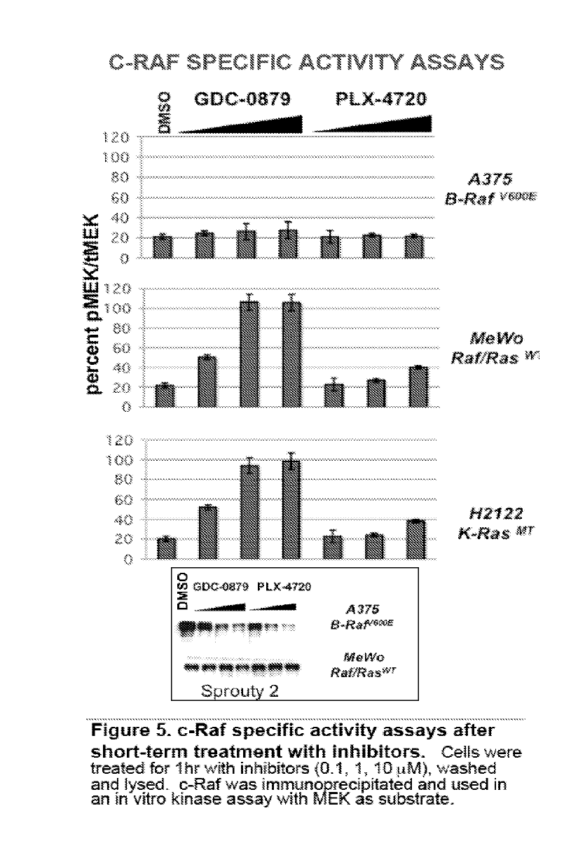

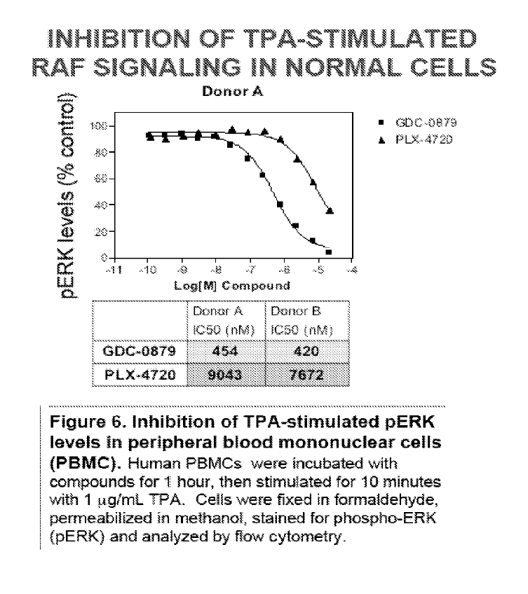

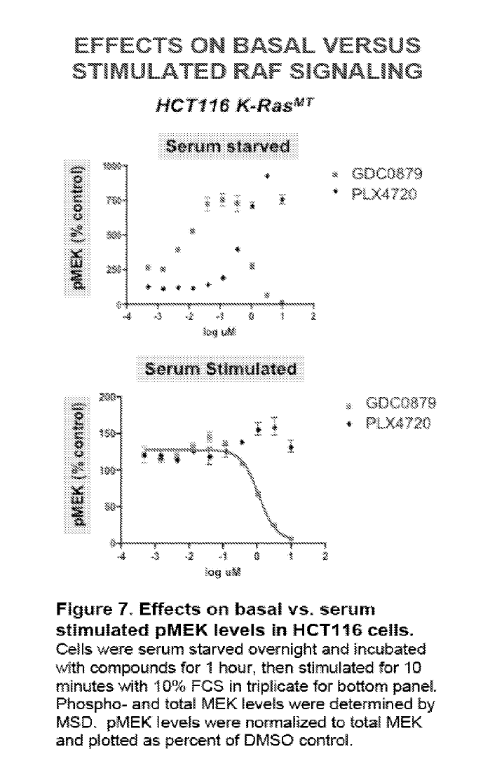

To understand the role of the Raf pathway with different mutations in the Ras and Raf genes, we characterized two selective small molecule Raf inhibitors with distinct potency profiles against wild-type (WT) B-Raf and c-Raf versus mutant (MT) B-Raf^{V600E}. Despite their biochemical differences, they had identical cellular profiles, being potent against B-Raf^{V600E} but not WT or Ras MT tumors. Both inhibitors induced activation of the Raf/MEK/ERK pathway in non-BRAF^{V600E} lines, via primarily the c-Raf isoform. In contrast, they inhibited phorbol ester and growth factor-stimulated Raf/MEK/ERK activity according to their predicted biochemical potencies. Thus, the cellular specificity of selective Raf inhibitors for B-Raf^{V600E} lines is not simply a reflection of their selectivity for the B-Raf^{V600E} isoform, but rather reflects the complex regulation of Raf activity in different cellular contexts. Biochemical selectivity for the B-Raf^{V600E} is not the only driver for cellular efficacy profiles of Raf inhibitors. Inhibitors induce pMEK levels selectively in non-V600E mutant lines through c-Raf. Inhibitors induce c-Raf specific activity and pMEK levels rapidly and in a dose-dependent manner according to their potency. Under basal conditions, bell-shaped curve for GDC-0879 suggests dual stimulatory vs. inhibitory effects on c-Raf. B- and c-Raf pathway status in different contexts determines Raf inhibitory pharmacodynamics. The results of the characterization are shown in FIGS. 1-7.

### Example 3

### Growth in Lung Tumor Xenografts after Dosing with B-RAF Inhibitor

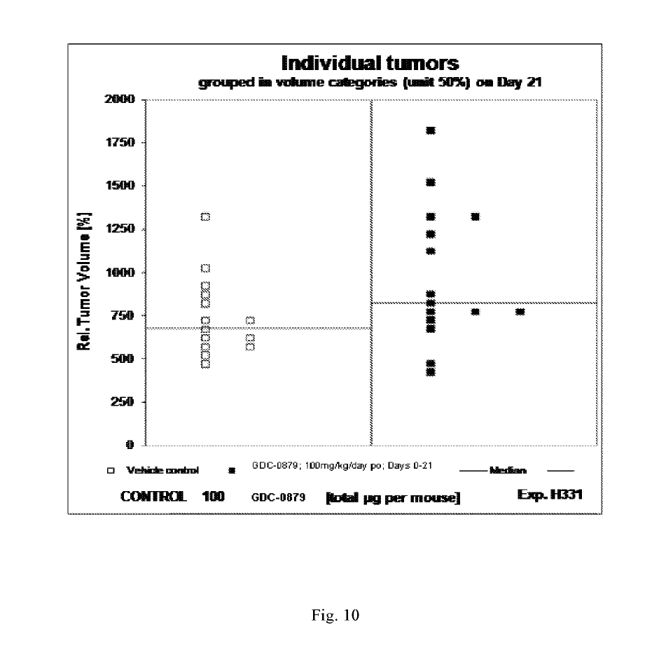

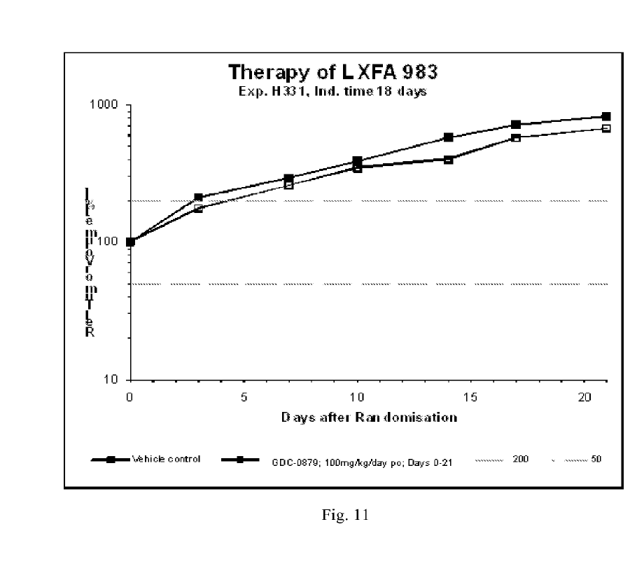

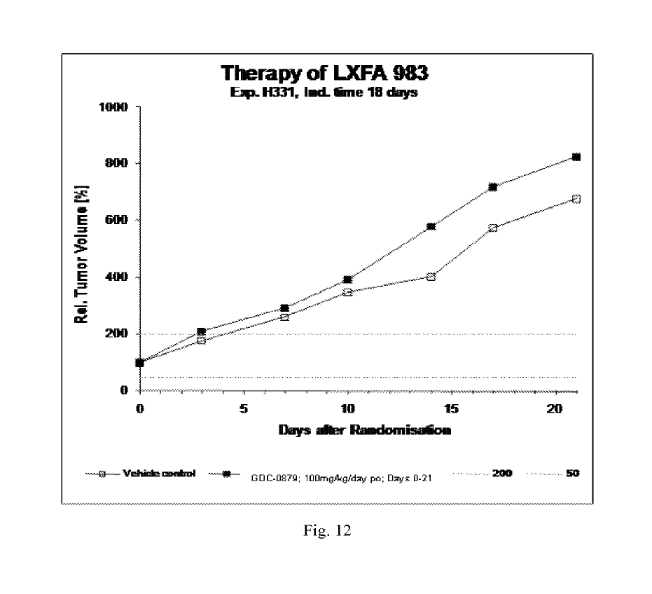

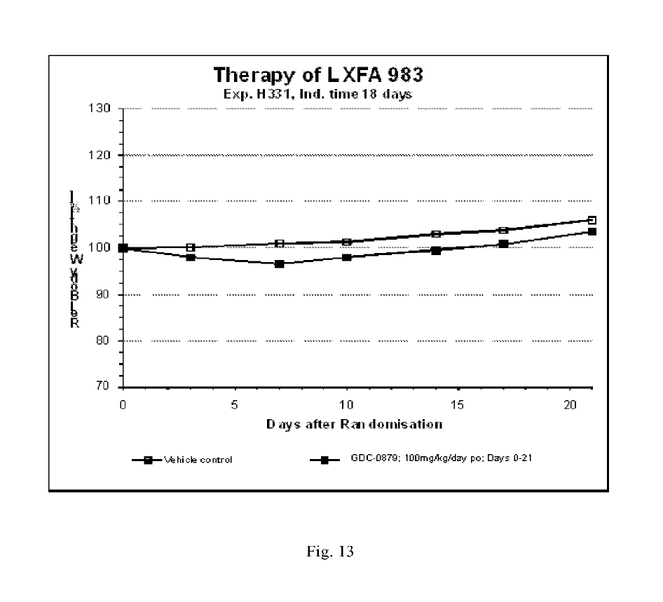

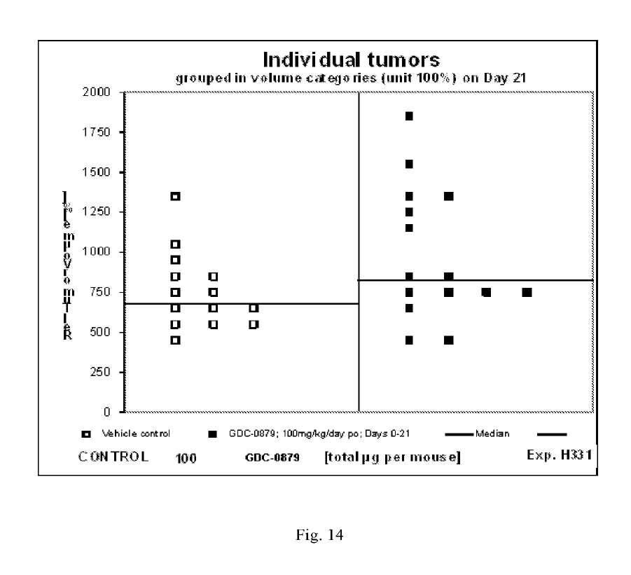

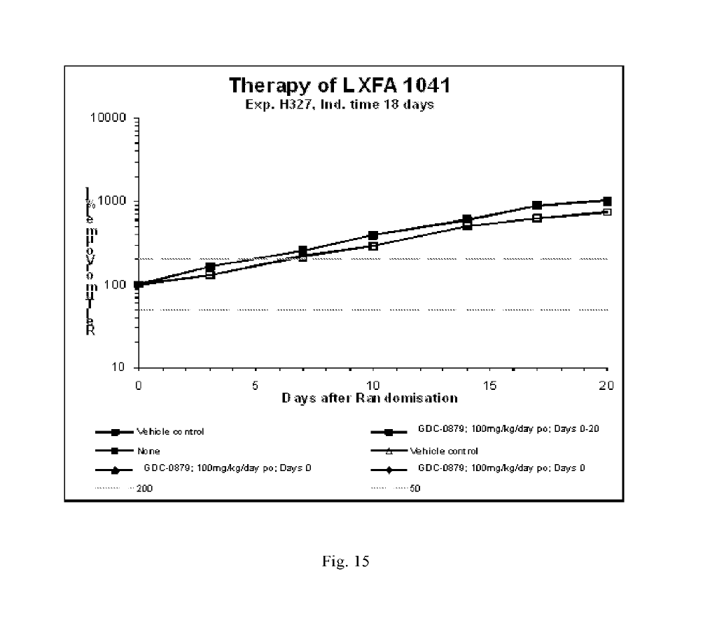

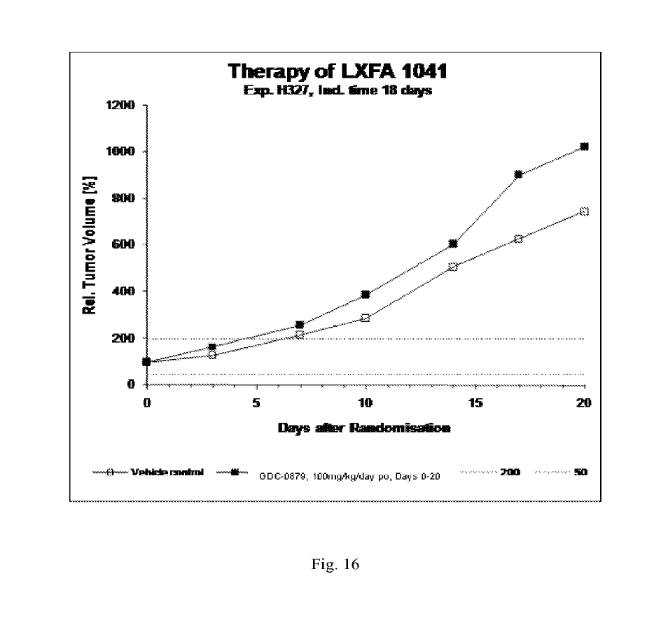

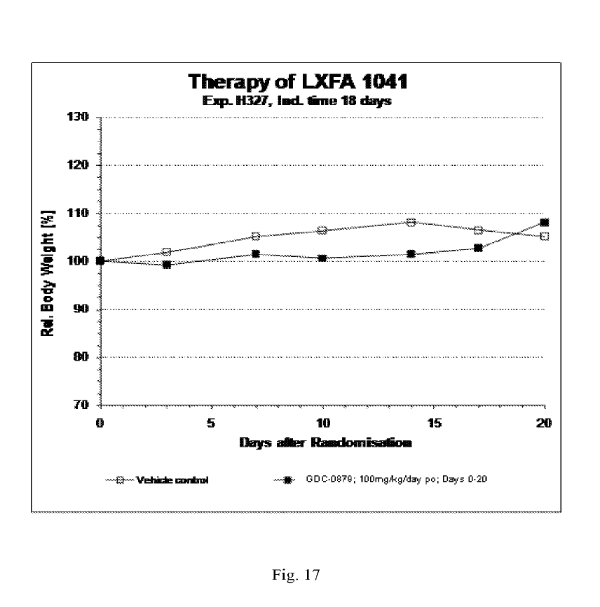

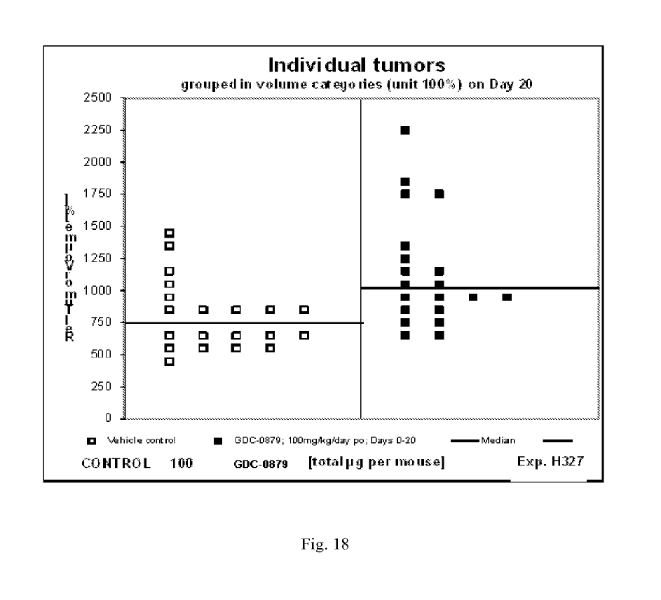

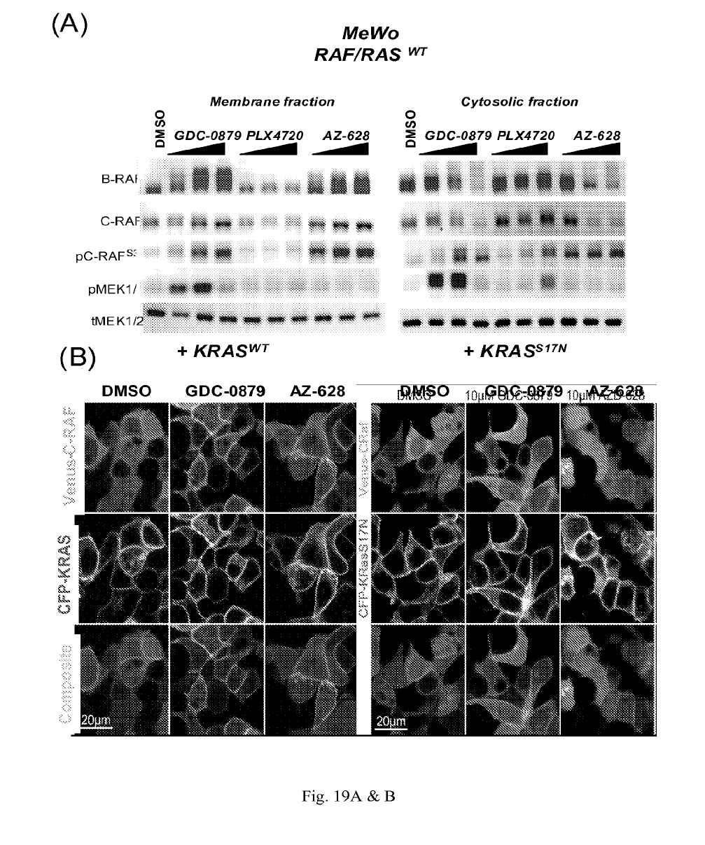

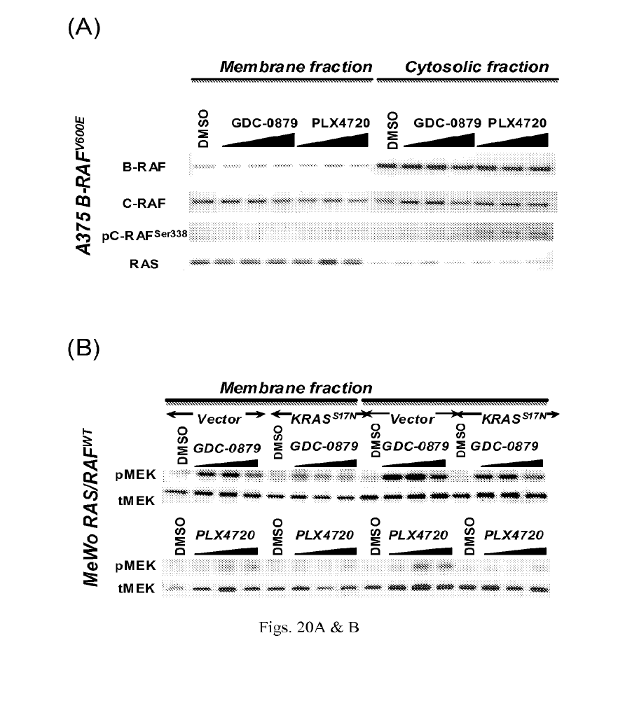

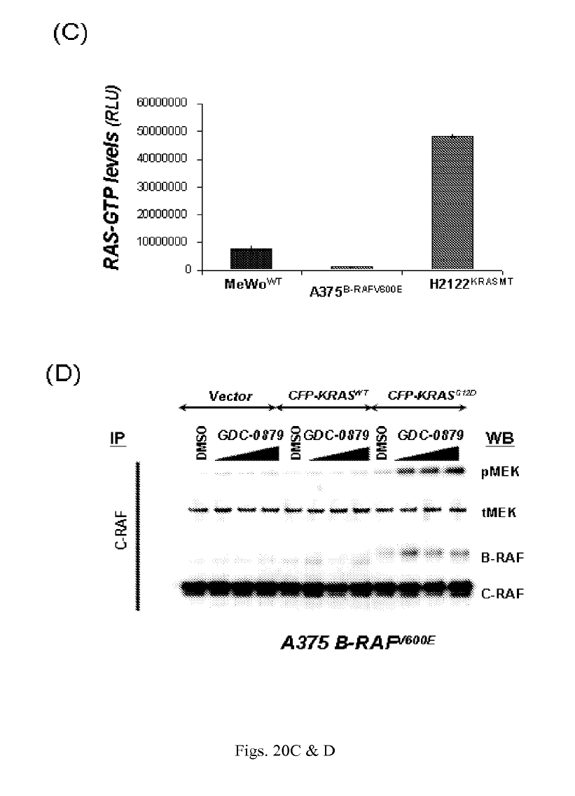

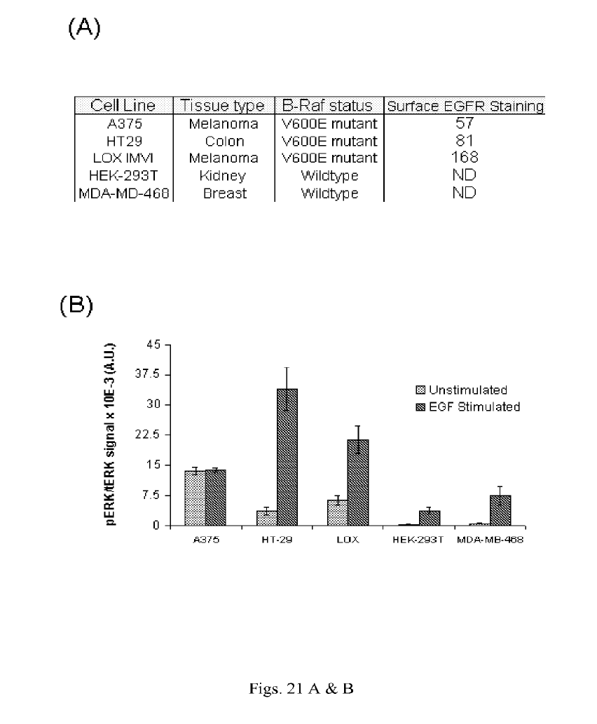

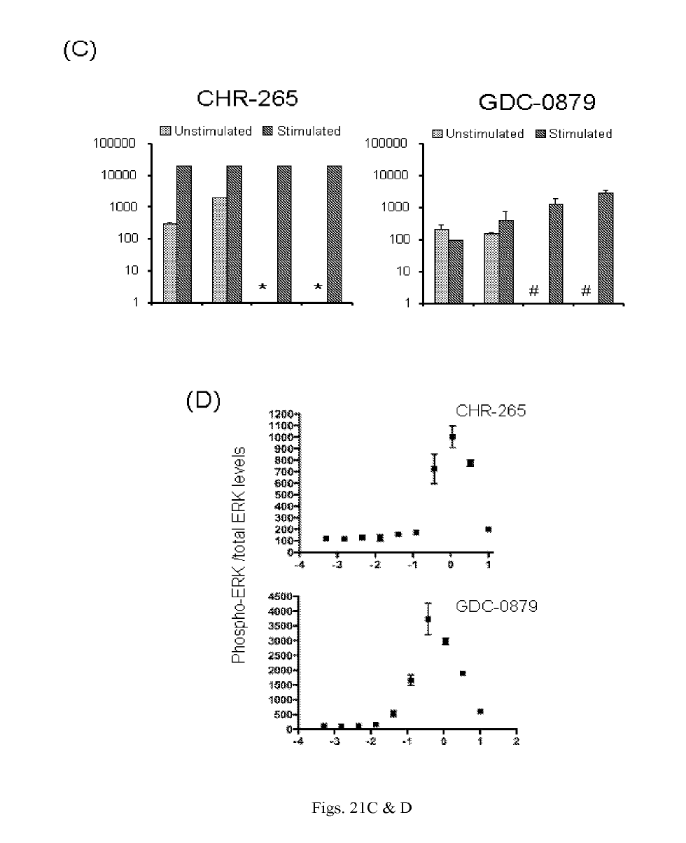

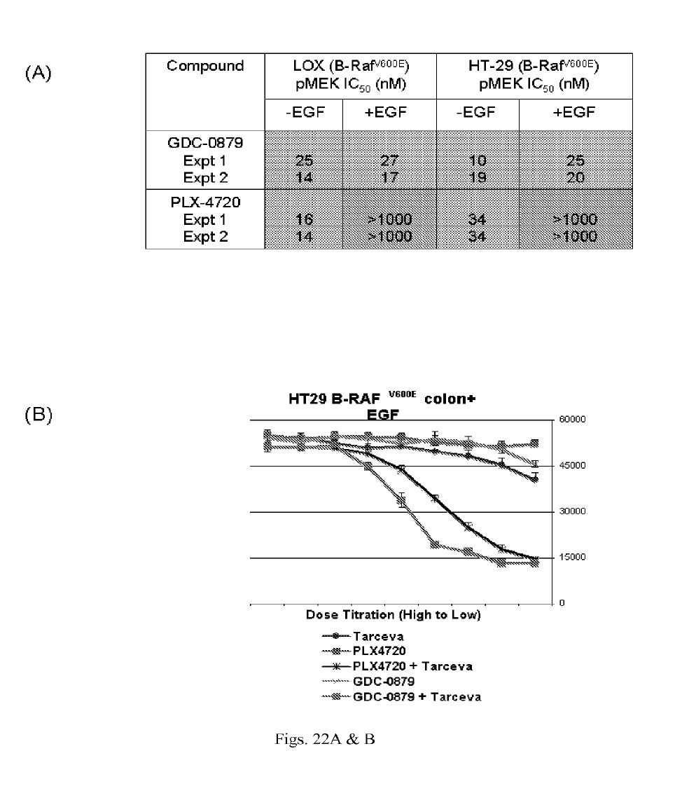

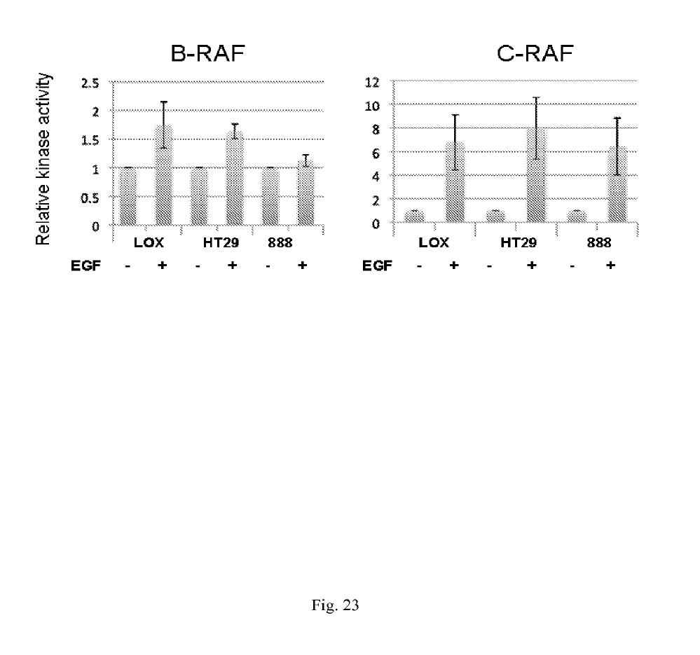

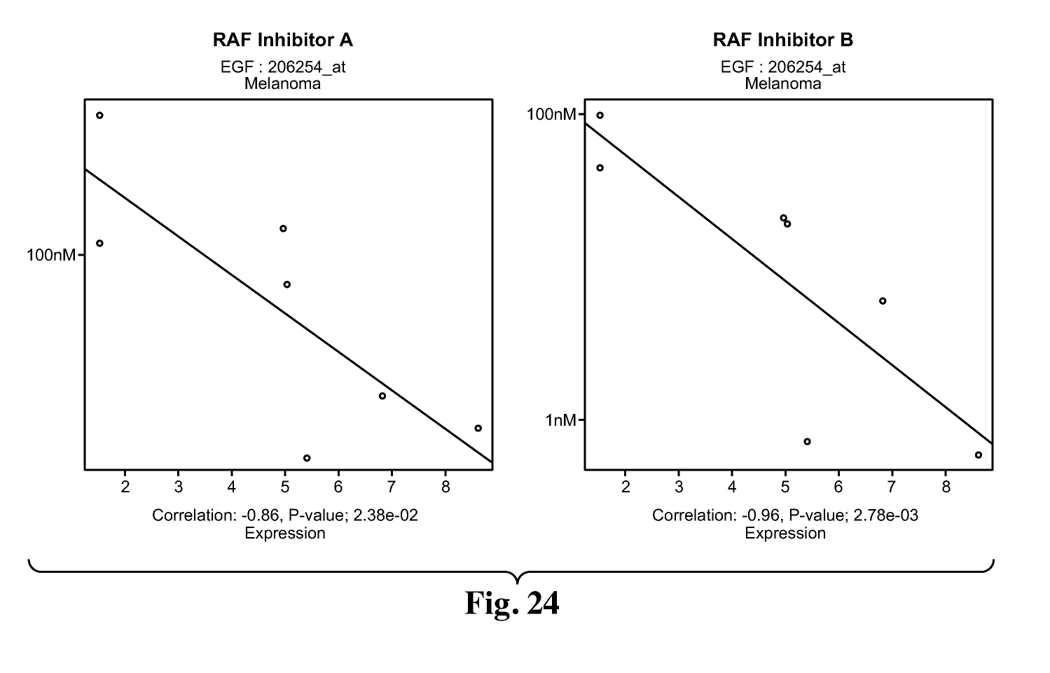

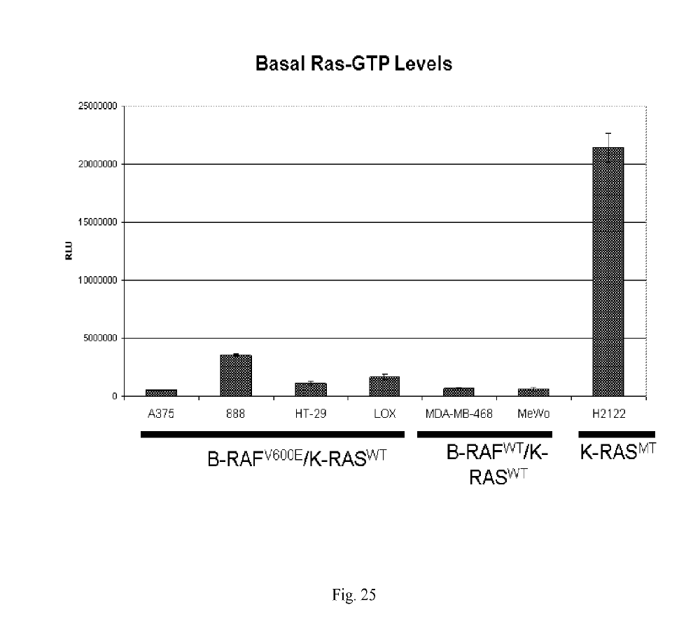

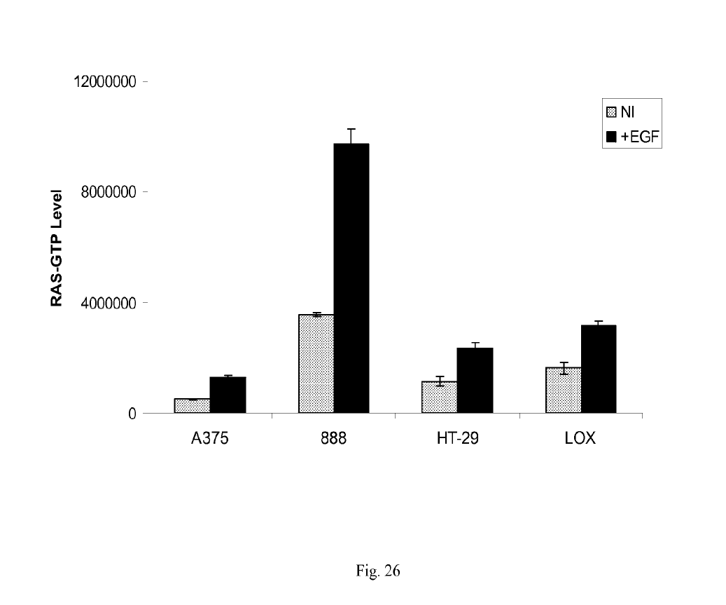

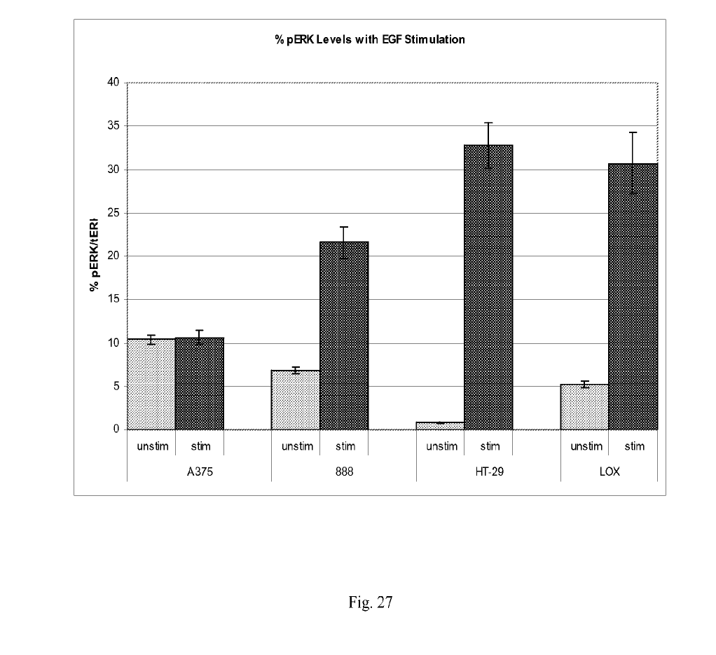

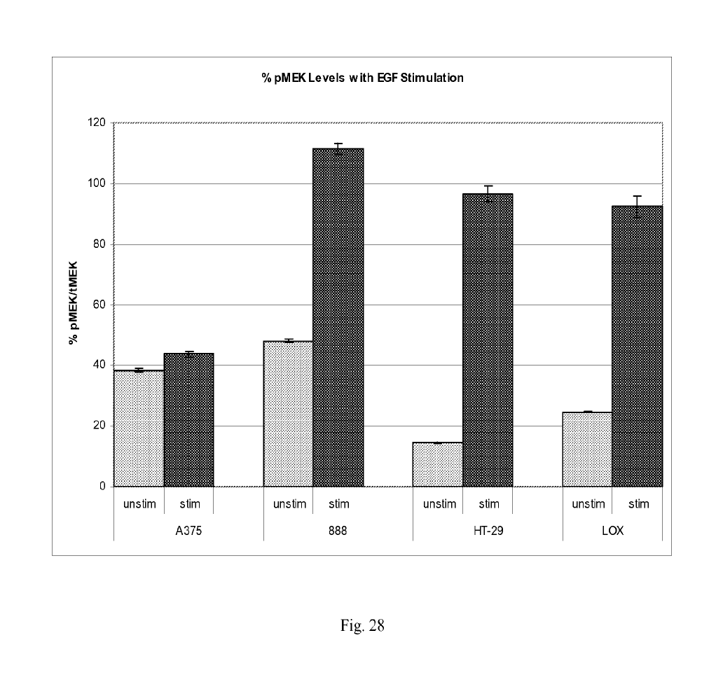

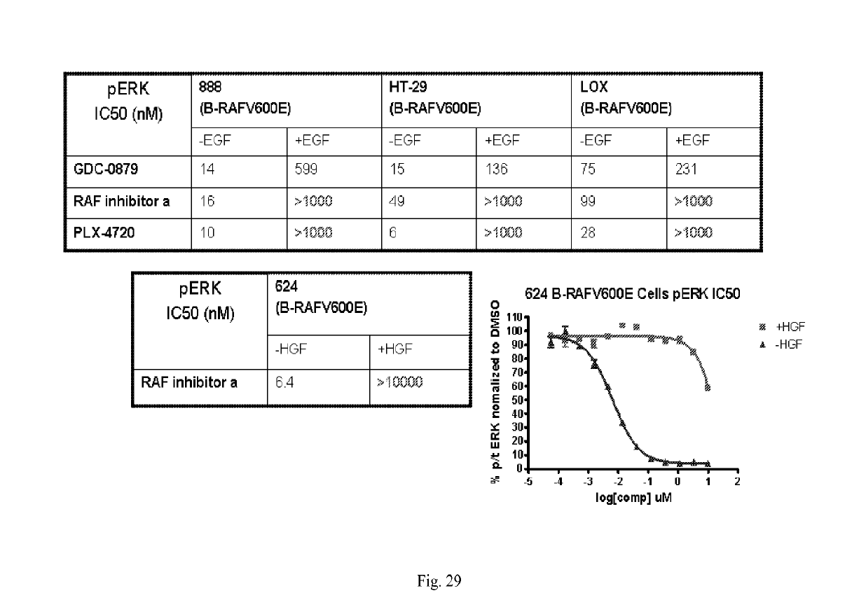

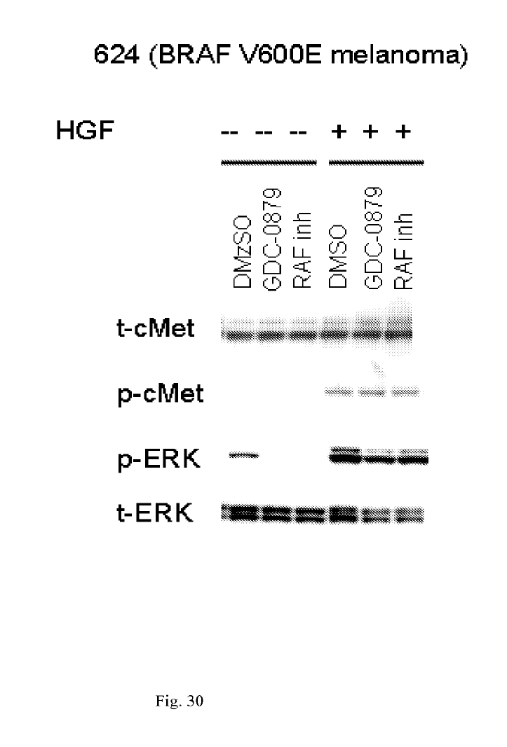

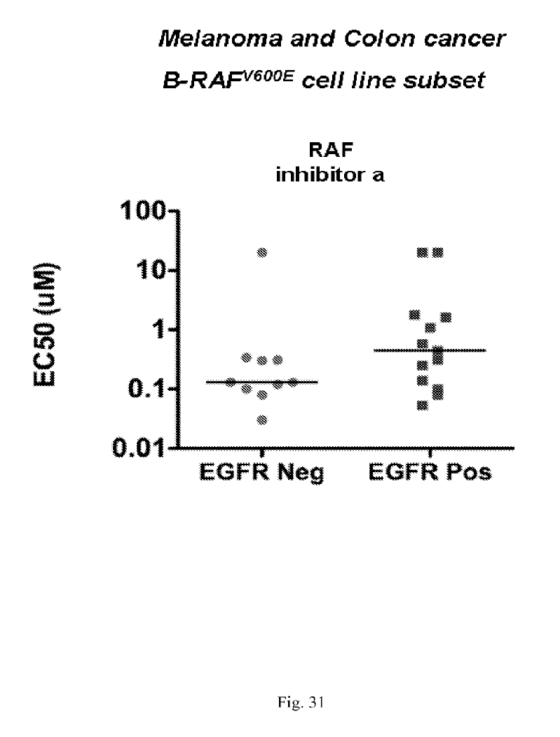

The data are shown below, and in FIGS. 10-14 for Experiment H331 and FIGS. 15-18 for Experiment H327.

| Oncotest Exp-Nr. | H331-1 | | | | Implant/Rando/Induction time | | | | | 18 days | | | | | |
|---|---|---|---|---|---|---|---|---|---|---|---|---|---|---|---|
| Tumortyp Nr./Pass | LXFA 983/9N4 | | | | End result/last study day | | | | | 21 | | | | | |
| Tumor Model | Lung, adeno | | | | Therapy | | | | | Vehicle control; 10 ml/kg; Days 0-21 | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | 3 | 7 | 10 | 14 | 17 | 21 | [days] | 0 | 3 | 7 | 10 | 14 | 17 | 21 |
| 532 r | 45.6 | 80.1 | 115.2 | 173.8 | 183.3 | 251.3 | 281.6 | >22 | 27.5 | 27.7 | 27.0 | 27.6 | 29.0 | 29.7 | 30.2 |
| 532 l | 111.6 | 275.7 | 415.2 | 540.7 | 957.6 | 1264.7 | 1454.6 | | | | | | | | |
| 1328 r | 86.2 | 139.3 | 292.8 | 392.9 | 540.0 | 589.8 | 708.7 | >21 | 26.4 | 26.5 | 27.2 | 27.5 | 28.7 | 29.4 | 28.7 |
| 1341 r | 137.3 | 178.9 | 302.6 | 354.8 | 491.9 | 804.8 | 1019.6 | >21 | 23.6 | 23.6 | 24.0 | 23.8 | 24.0 | 24.0 | 24.6 |
| 1342 r | 97.5 | 176.4 | 210.5 | 419.8 | 393.4 | 437.5 | 540.6 | >21 | 27.5 | 27.4 | 28.1 | 27.5 | 28.0 | 27.7 | 28.8 |
| 1344 r | 226.5 | 405.0 | 657.3 | 713.9 | 659.8 | 879.5 | 1140.8 | >21 | 27.8 | 28.3 | 30.5 | 30.7 | 29.1 | 29.4 | 29.8 |
| 1348 r | 148.1 | 196.0 | 343.2 | 445.5 | 563.1 | 770.2 | 919.6 | >21 | 26.9 | 26.0 | 25.9 | 26.0 | 26.6 | 25.9 | 26.8 |
| 1348 l | 134.5 | 246.4 | 421.2 | 514.4 | 715.6 | 749.9 | 881.0 | | | | | | | | |
| 1352 r | 143.7 | 239.1 | 288.0 | 361.3 | 384.6 | 465.5 | 689.1 | >21 | 28.9 | 28.8 | 29.0 | 29.4 | 29.3 | 30.6 | 31.8 |
| 1352 l | 45.6 | 77.2 | 94.2 | 124.9 | 153.6 | 267.2 | 319.4 | | | | | | | | |
| 1361 r | 126.0 | 290.5 | 510.3 | 562.2 | 778.8 | 832.3 | 1166.4 | >21 | 28.3 | 28.6 | 28.9 | 29.0 | 30.0 | 29.3 | 29.6 |
| 1361 l | 55.3 | 111.6 | 147.9 | 147.9 | 194.2 | 310.4 | 320.0 | | | | | | | | |
| 1362 r | 65.0 | 126.0 | 156.3 | 221.8 | 426.5 | 472.0 | 553.0 | >22 | 26.8 | 26.0 | 26.4 | 26.3 | 26.5 | 26.3 | 26.1 |
| 1365 l | 50.6 | 77.1 | 141.5 | 178.9 | 389.3 | 514.6 | 525.0 | >22 | 24.3 | 24.4 | 24.0 | 24.9 | 25.3 | 25.3 | 26.1 |
| n | 14 | 14 | 14 | 14 | 14 | 14 | 14 | | 10 | 10 | 10 | 10 | 10 | 10 | 10 |
| Median | 104.5 | 177.6 | 290.4 | 377.1 | 459.2 | 552.2 | 698.9 | | 27.2 | 27.0 | 27.1 | 27.5 | 28.4 | 28.5 | 28.8 |
| MIN | 45.6 | 77.1 | 94.2 | 124.9 | 153.6 | 251.3 | 281.6 | | 23.6 | 23.6 | 24.0 | 23.8 | 24.0 | 24.0 | 24.6 |
| MAX | 226.5 | 405.0 | 657.3 | 713.9 | 957.6 | 1264.7 | 1454.6 | | 28.9 | 28.8 | 30.5 | 30.7 | 30.0 | 30.6 | 31.8 |
| 95% MIN | 86.2 | 139.3 | 210.5 | 354.8 | 384.6 | 465.5 | 553.0 | | 26.4 | 26.0 | 25.9 | 26.0 | 26.5 | 26.3 | 26.8 |
| 95% MAX | 134.5 | 239.1 | 343.2 | 445.5 | 563.1 | 770.2 | 919.6 | | 27.8 | 27.7 | 28.1 | 27.6 | 29.0 | 29.3 | 29.8 |
| | Relative tumor volume [%] | | | | | | | | Relative body weight [%] | | | | | | |
| Animal-No | 0 | 3 | 7 | 10 | 14 | 17 | 21 | | 0 | 3 | 7 | 10 | 14 | 17 | 21 |
| 532 r | 100.0 | 175.7 | 252.8 | 381.4 | 402.3 | 551.5 | 618.1 | >22 | 100.0 | 100.7 | 98.2 | 100.4 | 105.5 | 108.0 | 109.8 |
| 532 l | 100.0 | 247.0 | 372.0 | 484.5 | 858.1 | 1133.3 | 1303.4 | | | | | | | | |
| 1328 r | 100.0 | 161.5 | 339.6 | 455.7 | 626.4 | 684.2 | 822.1 | >21 | 100.0 | 100.4 | 103.0 | 104.2 | 108.7 | 111.4 | 108.7 |
| 1341 r | 100.0 | 130.3 | 220.4 | 258.4 | 358.2 | 586.1 | 742.5 | >21 | 100.0 | 100.0 | 101.7 | 100.8 | 101.7 | 101.7 | 104.2 |
| 1342 r | 100.0 | 181.0 | 216.0 | 430.7 | 403.6 | 448.8 | 554.6 | >21 | 100.0 | 99.6 | 102.2 | 100.0 | 101.8 | 100.7 | 104.7 |
| 1344 r | 100.0 | 178.8 | 290.2 | 315.2 | 291.3 | 388.3 | 503.7 | >21 | 100.0 | 101.8 | 109.7 | 110.4 | 104.7 | 105.8 | 107.2 |
| 1348 r | 100.0 | 132.3 | 231.7 | 300.8 | 380.2 | 520.0 | 620.9 | >21 | 100.0 | 96.7 | 96.3 | 96.7 | 98.9 | 96.3 | 99.6 |
| 1348 l | 100.0 | 183.2 | 313.1 | 382.4 | 531.9 | 557.4 | 654.8 | | | | | | | | |
| 1352 r | 100.0 | 166.4 | 200.5 | 251.5 | 267.7 | 324.0 | 479.7 | >21 | 100.0 | 99.7 | 100.3 | 101.7 | 101.4 | 105.9 | 110.0 |
| 1352 l | 100.0 | 169.5 | 206.8 | 274.2 | 337.1 | 586.4 | 701.0 | | | | | | | | |
| 1361 r | 100.0 | 230.6 | 405.0 | 446.2 | 618.1 | 660.6 | 925.7 | >21 | 100.0 | 101.1 | 102.1 | 102.5 | 106.0 | 103.5 | 104.6 |
| 1361 l | 100.0 | 201.8 | 267.4 | 267.4 | 351.2 | 561.3 | 578.7 | | | | | | | | |
| 1362 r | 100.0 | 193.8 | 240.5 | 341.2 | 656.1 | 726.2 | 850.7 | >22 | 100.0 | 97.0 | 98.5 | 98.1 | 98.9 | 98.1 | 97.4 |
| 1365 l | 100.0 | 152.2 | 279.6 | 353.3 | 769.1 | 1016.4 | 1037.0 | >22 | 100.0 | 100.4 | 98.8 | 102.5 | 104.1 | 104.1 | 107.4 |
| n | 14 | 14 | 14 | 14 | 14 | 14 | 14 | | 10 | 10 | 10 | 10 | 10 | 10 | 10 |
| Median | 100.0 | 177.3 | 260.1 | 347.3 | 402.9 | 573.7 | 677.9 | | 100.0 | 100.2 | 101.0 | 101.3 | 103.0 | 103.8 | 106.0 |
| MIN | 100.0 | 130.3 | 200.5 | 251.5 | 267.7 | 324.0 | 479.7 | | 100.0 | 96.7 | 96.3 | 96.7 | 98.9 | 96.3 | 97.4 |
| MAX | 100.0 | 247.0 | 405.0 | 484.5 | 858.1 | 1133.3 | 1303.4 | | 100.0 | 101.8 | 109.7 | 110.4 | 108.7 | 111.4 | 110.0 |
| 95% MIN | 100.0 | 161.5 | 240.5 | 315.2 | 402.3 | 520.0 | 618.1 | | 100.0 | 99.6 | 98.5 | 100.0 | 101.4 | 100.7 | 104.2 |
| 95% MAX | 100.0 | 193.8 | 290.2 | 382.4 | 531.9 | 726.2 | 850.7 | | 100.0 | 100.7 | 103.0 | 104.2 | 104.7 | 105.9 | 107.4 |
| Oncotest Exp-Nr. | H331-2 | | | | Implant/Rando/Induction time | | | | | 18 days | | | | | |
| Tumortyp Nr./Pass | LXFA 983/9N4 | | | | End result/last study day | | | | | 21 | | | | | |
| Tumor Model | Lung, adeno | | | | Therapy | | | | | G-026887; 100 mg/kg/day; Days 0-21 | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | 3 | 7 | 10 | 14 | 17 | 21 | [days] | 0 | 3 | 7 | 10 | 14 | 17 | 21 |
| 1330 r | 117.2 | 265.6 | 392.9 | 431.7 | 578.8 | 705.7 | 893.3 | >21 | 27.2 | 26.8 | 26.3 | 26.7 | 26.8 | 27.2 | 28.6 |
| 1330 l | 121.1 | 166.6 | 325.1 | 379.3 | 655.5 | 742.6 | 1083.3 | | | | | | | | |
| 1332 l | 94.1 | 188.5 | 261.6 | 329.5 | 545.9 | 701.1 | 715.5 | >21 | 26.9 | 27.2 | 27.4 | 26.9 | 27.2 | 27.6 | 27.5 |
| 1333 r | 137.3 | 239.1 | 445.5 | 539.0 | 574.3 | 841.5 | 1030.4 | >21 | 27.3 | 26.7 | 26.3 | 26.7 | 28.2 | 28.5 | 29.3 |
| 1336 r | 237.3 | 546.2 | 689.1 | 964.8 | 1411.2 | 1708.0 | 1961.1 | >21 | 26.1 | 25.6 | 25.7 | 25.6 | 25.5 | 25.8 | 26.9 |
| 1336 l | 62.5 | 176.4 | 289.1 | 404.0 | 581.0 | 642.0 | 772.8 | | | | | | | | |
| 1339 r | 80.2 | 210.9 | 348.2 | 612.5 | 1115.4 | 1229.4 | 1472.3 | >21 | 29.4 | 27.9 | 27.1 | 27.6 | 29.1 | 29.5 | 31.1 |
| 1339 l | 123.0 | 268.8 | 344.4 | 525.4 | 951.3 | 1327.1 | 1618.7 | | | | | | | | |
| 1346 r | 111.6 | 236.3 | 455.1 | 528.2 | 891.1 | 1247.1 | 1461.9 | >21 | 24.8 | 24.7 | 24.2 | 25.3 | 25.8 | 25.8 | 26.6 |
| 1346 l | 74.4 | 156.8 | 253.1 | 302.6 | 318.2 | 426.5 | 550.0 | | | | | | | | |
| 1349 r | 219.4 | 392.9 | 560.0 | 570.0 | 597.5 | 778.8 | 958.8 | >21 | 31.2 | 29.4 | 28.9 | 29.1 | 29.4 | 29.5 | 30.6 |
| 1350 r | 55.3 | 119.1 | 216.6 | 348.5 | 390.2 | 520.5 | 609.2 | >22 | 30.2 | 29.2 | 28.1 | 29.1 | 30.2 | 30.6 | 31.0 |
| 1350 l | 150.4 | 310.7 | 352.5 | 372.6 | 510.0 | 606.4 | 712.9 | | | | | | | | |
| 1358 r | 130.7 | 295.9 | 383.3 | 406.6 | 499.4 | 758.4 | 874.7 | >22 | 27.6 | 27.1 | 27.6 | 28.6 | 28.9 | 29.0 | 28.7 |
| 1366 r | 74.4 | 83.2 | 113.4 | 200.9 | 461.7 | 640.5 | 1125.0 | >22 | 31.2 | 29.6 | 29.3 | 28.6 | 30.3 | 30.7 | 31.6 |
| n | 15 | 15 | 15 | 15 | 15 | 15 | 15 | | 10 | 10 | 10 | 10 | 10 | 10 | 10 |
| Median | 117.2 | 236.3 | 348.2 | 406.6 | 578.8 | 742.6 | 958.8 | | 27.5 | 27.2 | 27.3 | 27.3 | 28.6 | 28.8 | 29.0 |
| MIN | 55.3 | 83.2 | 113.4 | 200.9 | 318.2 | 426.5 | 550.0 | | 24.8 | 24.7 | 24.2 | 25.3 | 25.5 | 25.8 | 26.6 |
| MAX | 237.3 | 546.2 | 689.1 | 964.8 | 1411.2 | 1708.0 | 1961.1 | | 31.2 | 29.6 | 29.3 | 29.1 | 30.3 | 30.7 | 31.6 |
| 95% MIN | 94.1 | 188.5 | 289.1 | 372.6 | 510.0 | 701.1 | 874.7 | | 26.9 | 26.7 | 26.3 | 26.7 | 27.2 | 27.2 | 28.6 |
| 95% MAX | 137.3 | 295.9 | 392.9 | 539.0 | 655.5 | 841.5 | 1125.0 | | 29.4 | 27.9 | 28.1 | 27.6 | 29.1 | 29.5 | 29.3 |
| | Relative tumor volume [%] | | | | | | | | Relative body weight [%] | | | | | | |
| Animal-No | 0 | 3 | 7 | 10 | 14 | 17 | 21 | | 0 | 3 | 7 | 10 | 14 | 17 | 21 |
| 1330 r | 100.0 | 226.6 | 335.2 | 368.3 | 493.8 | 602.1 | 762.1 | >21 | 100.0 | 98.5 | 96.7 | 98.2 | 98.5 | 100.0 | 105.1 |
| 1330 l | 100.0 | 137.6 | 268.5 | 313.3 | 541.3 | 613.3 | 894.7 | | | | | | | | |
| 1332 l | 100.0 | 200.4 | 278.0 | 350.2 | 580.3 | 745.2 | 760.5 | >21 | 100.0 | 101.1 | 101.9 | 100.0 | 101.1 | 102.6 | 102.2 |
| 1333 r | 100.0 | 174.1 | 324.4 | 392.5 | 418.3 | 612.8 | 750.4 | >21 | 100.0 | 97.8 | 96.3 | 97.8 | 103.3 | 104.4 | 107.3 |
| 1336 r | 100.0 | 230.2 | 290.4 | 406.6 | 594.7 | 719.8 | 826.5 | >21 | 100.0 | 98.1 | 98.5 | 98.1 | 97.7 | 98.9 | 103.1 |
| 1336 l | 100.0 | 282.2 | 462.6 | 646.4 | 929.7 | 1027.1 | 1236.5 | | | | | | | | |
| 1339 r | 100.0 | 263.0 | 434.2 | 763.8 | 1390.9 | 1533.1 | 1836.0 | >21 | 100.0 | 94.9 | 92.2 | 93.9 | 99.0 | 100.3 | 105.8 |
| 1339 l | 100.0 | 218.5 | 280.0 | 427.1 | 773.4 | 1078.9 | 1316.0 | | | | | | | | |
| 1346 r | 100.0 | 211.7 | 407.8 | 473.3 | 798.5 | 1117.5 | 1309.9 | >21 | 100.0 | 99.6 | 97.6 | 102.0 | 104.0 | 104.0 | 107.3 |
| 1346 l | 100.0 | 210.9 | 340.4 | 406.9 | 427.9 | 573.5 | 739.6 | | | | | | | | |
| 1349 r | 100.0 | 179.1 | 255.3 | 259.8 | 272.4 | 355.0 | 437.1 | >21 | 100.0 | 94.2 | 92.6 | 93.3 | 94.2 | 94.6 | 98.1 |
| 1350 r | 100.0 | 215.3 | 391.6 | 630.2 | 705.6 | 941.4 | 1101.7 | >22 | 100.0 | 96.7 | 93.0 | 96.4 | 100.0 | 101.3 | 102.6 |
| 1350 l | 100.0 | 206.6 | 234.4 | 247.8 | 339.1 | 403.2 | 474.0 | | | | | | | | |
| 1358 r | 100.0 | 226.4 | 293.3 | 311.1 | 382.1 | 580.3 | 669.2 | >22 | 100.0 | 98.2 | 100.0 | 103.6 | 104.7 | 105.1 | 104.0 |
| 1366 r | 100.0 | 111.8 | 152.3 | 269.9 | 620.2 | 860.5 | 1511.3 | >22 | 100.0 | 94.9 | 93.9 | 91.7 | 97.1 | 98.4 | 101.3 |
| n | 15 | 15 | 15 | 15 | 15 | 15 | 15 | | 10 | 10 | 10 | 10 | 10 | 10 | 10 |
| Median | 100.0 | 211.7 | 293.3 | 392.5 | 580.3 | 719.8 | 826.5 | | 100.0 | 97.9 | 96.5 | 97.9 | 99.5 | 100.8 | 103.5 |
| MIN | 100.0 | 111.8 | 152.3 | 247.8 | 272.4 | 355.0 | 437.1 | | 100.0 | 94.2 | 92.2 | 91.7 | 94.2 | 94.6 | 98.1 |
| MAX | 100.0 | 282.2 | 462.6 | 763.8 | 1390.9 | 1533.1 | 1836.0 | | 100.0 | 101.1 | 101.9 | 103.6 | 104.7 | 105.1 | 107.3 |
| 95% MIN | 100.0 | 200.4 | 278.0 | 350.2 | 493.8 | 612.8 | 760.5 | | 100.0 | 96.7 | 96.3 | 96.4 | 97.7 | 98.9 | 102.2 |
| 95% MAX | 100.0 | 230.2 | 340.4 | 473.3 | 773.4 | 941.4 | 1101.7 | | 100.0 | 98.5 | 98.5 | 100.0 | 101.1 | 102.6 | 105.1 |
| Oncotest Exp-Nr. | H331-3 | | | | Implant/Rando/Induction time | | | | | 18 days | | | | | |
| Tumortyp Nr./Pass | LXFA 983/9N4 | | | | End result/last study day | | | | | 21 | | | | | |
| Tumor Model | Lung, adeno | | | | Therapy | | | | | None | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | | | | | | | [days] | | | | 0 | | | |
| 1335 r | 113.1 | | | | | | | >0 | | | | 23.0 | | | |
| 1359 l | 71.7 | | | | | | | >0 | | | | 24.1 | | | |
| 1364 r | 77.1 | | | | | | | >0 | | | | 22.5 | | | |
| n | 3 | | | | | | | | | | | 3 | | | |
| Median | 77.1 | | | | | | | | | | | 23.0 | | | |
| MIN | 71.7 | | | | | | | | | | | 22.5 | | | |
| MAX | 113.1 | | | | | | | | | | | 24.1 | | | |
| 95% MIN | 71.7 | | | | | | | | | | | 22.5 | | | |
| 95% MAX | 113.1 | | | | | | | | | | | 24.1 | | | |
| | Relative tumor volume [%] | | | | | | | | Relative body weight [%] | | | | | | |
| Animal-No | 0 | | | | | | | | | | | 0 | | | |
| 1335 r | 100.0 | | | | | | | >0 | | | | 100.0 | | | |
| 1359 l | 100.0 | | | | | | | >0 | | | | 100.0 | | | |
| 1364 r | 100.0 | | | | | | | >0 | | | | 100.0 | | | |
| n | 3 | | | | | | | | | | | 3 | | | |
| Median | 100.0 | | | | | | | | | | | 100.0 | | | |
| MIN | 100.0 | | | | | | | | | | | 100.0 | | | |
| MAX | 100.0 | | | | | | | | | | | 100.0 | | | |
| 95% MIN | 100.0 | | | | | | | | | | | 100.0 | | | |
| 95% MAX | 100.0 | | | | | | | | | | | 100.0 | | | |
| Oncotest Exp-Nr. | H331-4 | | | | Implant/Rando/Induction time | | | | | 18 days | | | | | |
| Tumortyp Nr./Pass | LXFA 983/9N4 | | | | End result/last study day | | | | | 21 | | | | | |
| Tumor Model | Lung, adeno | | | | Therapy | | | | | Vehicle control; 10 ml/kg; Day 0 | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | | | | | | | [days] | | | | 0 | | | |
| 1326 l | 384.8 | | | | | | | >0 | | | | 30.5 | | | |
| 1329 r | 156.3 | | | | | | | >0 | | | | 28.3 | | | |
| 1338 l | 157.7 | | | | | | | >0 | | | | 24.2 | | | |
| 1345 r | 252.0 | | | | | | | >0 | | | | 28.0 | | | |
| 1347 r | 171.1 | | | | | | | >0 | | | | 29.3 | | | |
| 1353 l | 179.6 | | | | | | | >0 | | | | 29.0 | | | |
| n | 6 | | | | | | | | | | | 6 | | | |
| Median | 175.3 | | | | | | | | | | | 28.7 | | | |
| MIN | 156.3 | | | | | | | | | | | 24.2 | | | |
| MAX | 384.8 | | | | | | | | | | | 30.5 | | | |
| 95% MIN | 156.3 | | | | | | | | | | | 28.0 | | | |
| 95% MAX | 252.0 | | | | | | | | | | | 29.3 | | | |
| | Relative tumor volume [%] | | | | | | | | Relative body weight [%] | | | | | | |
| Animal-No | 0 | | | | | | | | | | | 0 | | | |
| 1326 l | 100.0 | | | | | | | >0 | | | | 100.0 | | | |
| 1329 r | 100.0 | | | | | | | >0 | | | | 100.0 | | | |
| 1338 l | 100.0 | | | | | | | >0 | | | | 100.0 | | | |
| 1345 r | 100.0 | | | | | | | >0 | | | | 100.0 | | | |
| 1347 r | 100.0 | | | | | | | >0 | | | | 100.0 | | | |
| 1353 l | 100.0 | | | | | | | >0 | | | | 100.0 | | | |
| n | 6 | | | | | | | | | | | 6 | | | |
| Median | 100.0 | | | | | | | | | | | 100.0 | | | |
| MIN | 100.0 | | | | | | | | | | | 100.0 | | | |
| MAX | 100.0 | | | | | | | | | | | 100.0 | | | |
| 95% MIN | 100.0 | | | | | | | | | | | 100.0 | | | |
| 95% MAX | 100.0 | | | | | | | | | | | 100.0 | | | |
| Oncotest Exp-Nr. | H331-5 | | | | Implant/Rando/Induction time | | | | | 18 days | | | | | |
| Tumortyp Nr./Pass | LXFA 983/9N4 | | | | End result/last study day | | | | | 21 | | | | | |
| Tumor Model | Lung, adeno | | | | Therapy | | | | | G-026887; 100 mg/kg/day po; Day 0 | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | | | | | | | [days] | | | | 0 | | | |
| 1354 r | 449.6 | | | | | | | >0 | | | | 26.5 | | | |
| 1355 l | 188.4 | | | | | | | >0 | | | | 27.2 | | | |
| 1356 r | 226.8 | | | | | | | >0 | | | | 27.3 | | | |
| 1357 r | 231.2 | | | | | | | >0 | | | | 29.3 | | | |
| 1359 r | 196.6 | | | | | | | >0 | | | | 28.1 | | | |
| 1367 r | 287.6 | | | | | | | >0 | | | | 25.5 | | | |
| n | 6 | | | | | | | | | | | 6 | | | |
| Median | 229.0 | | | | | | | | | | | 27.3 | | | |
| MIN | 188.4 | | | | | | | | | | | 25.5 | | | |
| MAX | 449.6 | | | | | | | | | | | 29.3 | | | |
| 95% MIN | 188.4 | | | | | | | | | | | 26.5 | | | |
| 95% MAX | 287.6 | | | | | | | | | | | 28.1 | | | |
| | Relative tumor volume [%] | | | | | | | | Relative body weight [%] | | | | | | |
| Animal-No | 0 | | | | | | | | | | | 0 | | | |
| 1354 r | 100.0 | | | | | | | >0 | | | | 100.0 | | | |
| 1355 l | 100.0 | | | | | | | >0 | | | | 100.0 | | | |
| 1356 r | 100.0 | | | | | | | >0 | | | | 100.0 | | | |
| 1357 r | 100.0 | | | | | | | >0 | | | | 100.0 | | | |
| 1359 r | 100.0 | | | | | | | >0 | | | | 100.0 | | | |
| 1367 r | 100.0 | | | | | | | >0 | | | | 100.0 | | | |
| n | 6 | | | | | | | | | | | 6 | | | |
| Median | 100.0 | | | | | | | | | | | 100.0 | | | |
| MIN | 100.0 | | | | | | | | | | | 100.0 | | | |
| MAX | 100.0 | | | | | | | | | | | 100.0 | | | |
| 95% MIN | 100.0 | | | | | | | | | | | 100.0 | | | |
| 95% MAX | 100.0 | | | | | | | | | | | 100.0 | | | |
| Oncotest Exp-Nr. | H331-6 | | | | Implant/Rando/Induction time | | | | | 18 days | | | | | |
| Tumortyp Nr./Pass | LXFA 983/9N4 | | | | End result/last study day | | | | | 21 | | | | | |
| Tumor Model | Lung, adeno | | | | Therapy | | | | | G-026887; 100 mg/kg/day po; Day 0 | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | | | | | | | [days] | | | | 0 | | | |
| 1334 r | 505.4 | | | | | | | >0 | | | | 27.2 | | | |
| 1337 r | 249.4 | | | | | | | >0 | | | | 27.9 | | | |
| 1343 r | 326.4 | | | | | | | >0 | | | | 25.6 | | | |
| n | 3 | | | | | | | | | | | 3 | | | |
| Median | 326.4 | | | | | | | | | | | 27.2 | | | |
| MIN | 249.4 | | | | | | | | | | | 25.6 | | | |
| MAX | 505.4 | | | | | | | | | | | 27.9 | | | |
| 95% MIN | 249.4 | | | | | | | | | | | 25.6 | | | |
| 95% MAX | 505.4 | | | | | | | | | | | 27.9 | | | |
| | Relative tumor volume [%] | | | | | | | | Relative body weight [%] | | | | | | |
| Animal-No | 0 | | | | | | | | | | | 0 | | | |
| 1334 r | 100.0 | | | | | | | >0 | | | | 100.0 | | | |
| 1337 r | 100.0 | | | | | | | >0 | | | | 100.0 | | | |
| 1343 r | 100.0 | | | | | | | >0 | | | | 100.0 | | | |
| n | 3 | | | | | | | | | | | 3 | | | |
| Median | 100.0 | | | | | | | | | | | 100.0 | | | |
| MIN | 100.0 | | | | | | | | | | | 100.0 | | | |
| MAX | 100.0 | | | | | | | | | | | 100.0 | | | |
| 95% MIN | 100.0 | | | | | | | | | | | 100.0 | | | |
| 95% MAX | 100.0 | | | | | | | | | | | 100.0 | | | |

| Oncotest Exp-Nr. | H327-1 | | | | Implant/Rando/Induction time | | | | 18 days | | | | | | |
|---|---|---|---|---|---|---|---|---|---|---|---|---|---|---|---|
| Tumortyp Nr./Pass | LXFA 1041/9N4 | | | | End result/last study day | | | | 20 | | | | | | |
| Tumor Model | Lung, adeno | | | | Therapy | | | | Vehicle control; 10 ml/kg; Days 0-20 | | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | 3 | 7 | 10 | 14 | 17 | 20 | [days] | 0 | 3 | 7 | 10 | 14 | 17 | 20 |
| 1237 r | 90.9 | 100.9 | 207.8 | 327.2 | 690.0 | 754.7 | 870.8 | >20 | 26.9 | 27.6 | 28.9 | 28.6 | 29.0 | 28.9 | 29.1 |
| 1237 l | 47.6 | 90.8 | 144.9 | 233.4 | 369.8 | 593.0 | 634.6 | | | | | | | | |
| 1238 r | 84.7 | 109.3 | 156.8 | 263.8 | 363.1 | 480.3 | 546.0 | >20 | 27.1 | 27.6 | 29.0 | 29.5 | 29.7 | 29.1 | 29.4 |
| 1238 l | 133.2 | 170.0 | 290.2 | 352.0 | 887.3 | 1009.8 | 1173.6 | | | | | | | | |
| 1242 r | 87.7 | 159.0 | 334.1 | 425.3 | 705.7 | 876.1 | 1032.2 | >20 | 27.0 | 26.9 | 27.6 | 27.3 | 27.5 | 27.4 | 27.5 |
| 1242 l | 47.6 | 74.4 | 137.3 | 213.2 | 458.8 | 637.6 | 700.1 | | | | | | | | |
| 1243 r | 65.0 | 86.2 | 141.5 | 150.0 | 329.5 | 481.6 | 523.5 | >20 | 27.7 | 28.6 | 29.1 | 30.4 | 31.5 | 31.9 | 31.5 |
| 1243 l | 60.0 | 87.7 | 166.1 | 210.5 | 367.8 | 535.0 | 540.0 | | | | | | | | |
| 1245 r | 115.2 | 210.9 | 288.0 | 356.4 | 464.8 | 596.8 | 733.1 | >20 | 30.2 | 30.8 | 31.8 | 32.5 | 33.3 | 31.9 | 27.2 |
| 1245 l | 90.8 | 109.3 | 172.8 | 212.5 | 331.6 | 504.2 | 525.0 | | | | | | | | |
| 1247 r | 113.4 | 147.9 | 225.0 | 341.0 | 517.6 | 717.9 | 777.3 | >20 | 24.8 | 26.1 | 26.5 | 26.4 | 26.9 | 26.6 | 27.0 |
| 1247 l | 62.5 | 80.1 | 139.3 | 173.8 | 316.9 | 347.9 | 422.3 | | | | | | | | |
| 1248 r | 111.6 | 117.0 | 202.2 | 268.8 | 396.9 | 455.5 | 572.2 | >20 | 26.5 | 27.7 | 28.7 | 28.5 | 29.5 | 29.3 | 29.9 |
| 1248 l | 90.8 | 90.8 | 186.2 | 193.6 | 293.5 | 351.0 | 408.7 | | | | | | | | |
| 1250 r | 62.5 | 68.8 | 95.3 | 152.1 | 394.3 | 482.2 | 549.8 | >21 | 25.9 | 25.4 | 25.6 | 25.2 | 25.4 | 24.9 | 25.0 |
| 1250 l | 113.4 | 141.5 | 231.0 | 295.9 | 615.3 | 1137.8 | 1195.3 | | | | | | | | |
| 1253 r | 80.2 | 109.8 | 207.8 | 213.6 | 336.0 | 387.2 | 405.0 | >21 | 27.0 | 27.4 | 27.7 | 27.6 | 28.0 | 27.8 | 27.2 |
| 1253 l | 94.2 | 147.9 | 258.6 | 281.6 | 397.8 | 505.0 | 560.0 | | | | | | | | |
| 1279 r | 191.1 | 261.6 | 378.9 | 653.1 | 950.6 | 1190.9 | 1576.9 | >21 | 28.1 | 28.0 | 28.6 | 28.3 | 28.9 | 28.8 | 28.7 |
| 1279 l | 68.8 | 83.1 | 111.6 | 152.1 | 361.3 | 395.7 | 416.3 | | | | | | | | |
| n | 20 | 20 | 20 | 20 | 20 | 20 | 20 | | 10 | 10 | 10 | 10 | 10 | 10 | 10 |
| Median | 89.2 | 109.3 | 194.2 | 248.6 | 395.6 | 520.0 | 566.1 | | 27.0 | 27.6 | 28.7 | 28.4 | 29.0 | 28.9 | 28.1 |
| MIN | 47.6 | 68.8 | 95.3 | 150.0 | 293.5 | 347.9 | 405.0 | | 24.8 | 25.4 | 25.6 | 25.2 | 25.4 | 24.9 | 25.0 |
| MAX | 191.1 | 261.6 | 378.9 | 653.1 | 950.6 | 1190.9 | 1576.9 | | 30.2 | 30.8 | 31.8 | 32.5 | 33.3 | 31.9 | 31.5 |
| 95% MIN | 80.2 | 100.9 | 172.8 | 233.4 | 394.3 | 504.2 | 572.2 | | 26.5 | 26.9 | 27.6 | 27.3 | 27.5 | 27.4 | 27.0 |
| 95% MAX | 94.2 | 141.5 | 231.0 | 327.2 | 517.6 | 717.9 | 777.3 | | 28.1 | 28.6 | 29.1 | 29.5 | 29.7 | 29.3 | 29.4 |
| | Relative tumor volume [%] | | | | | | | | Relative body weight [%] | | | | | | |
| Animal-No | 0 | 3 | 7 | 10 | 14 | 17 | 20 | | 0 | 3 | 7 | 10 | 14 | 17 | 20 |
| 1237 r | 100.0 | 111.0 | 228.5 | 359.8 | 758.7 | 829.8 | 957.5 | >20 | 100.0 | 102.6 | 107.4 | 106.3 | 107.8 | 107.4 | 108.2 |
| 1237 l | 100.0 | 190.7 | 304.4 | 490.5 | 777.1 | 1246.0 | 1333.6 | | | | | | | | |
| 1238 r | 100.0 | 129.1 | 185.1 | 311.5 | 428.7 | 567.1 | 644.6 | >20 | 100.0 | 101.8 | 107.0 | 108.9 | 109.6 | 107.4 | 108.5 |
| 1238 l | 100.0 | 127.6 | 217.9 | 264.3 | 666.1 | 758.1 | 881.1 | | | | | | | | |
| 1242 r | 100.0 | 181.2 | 380.9 | 484.8 | 804.4 | 998.7 | 1176.6 | >20 | 100.0 | 99.6 | 102.2 | 101.1 | 101.9 | 101.5 | 101.9 |
| 1242 l | 100.0 | 156.3 | 288.5 | 447.9 | 964.1 | 1339.8 | 1471.2 | | | | | | | | |
| 1243 r | 100.0 | 132.6 | 217.8 | 230.8 | 506.9 | 740.9 | 805.3 | >20 | 100.0 | 103.2 | 105.1 | 109.7 | 113.7 | 115.2 | 113.7 |
| 1243 l | 100.0 | 146.1 | 276.7 | 350.7 | 612.8 | 891.3 | 899.6 | | | | | | | | |
| 1245 r | 100.0 | 183.1 | 250.0 | 309.4 | 403.5 | 518.0 | 636.4 | >20 | 100.0 | 102.0 | 105.3 | 107.6 | 110.3 | 105.6 | 90.1 |
| 1245 l | 100.0 | 120.5 | 190.4 | 234.2 | 365.4 | 555.6 | 578.5 | | | | | | | | |
| 1247 r | 100.0 | 130.4 | 198.4 | 300.7 | 456.4 | 633.0 | 685.4 | >20 | 100.0 | 105.2 | 106.9 | 106.5 | 108.5 | 107.3 | 108.9 |
| 1247 l | 100.0 | 128.1 | 222.8 | 278.0 | 507.0 | 556.6 | 675.7 | | | | | | | | |
| 1248 r | 100.0 | 104.8 | 181.2 | 240.9 | 355.6 | 408.1 | 512.7 | >20 | 100.0 | 104.5 | 108.3 | 107.5 | 111.3 | 110.6 | 112.8 |
| 1248 l | 100.0 | 100.0 | 205.2 | 213.3 | 323.4 | 386.8 | 450.4 | | | | | | | | |
| 1250 r | 100.0 | 110.0 | 152.5 | 243.4 | 630.8 | 771.6 | 879.7 | >21 | 100.0 | 98.1 | 98.8 | 97.3 | 98.1 | 96.1 | 96.5 |
| 1250 l | 100.0 | 124.8 | 203.7 | 260.9 | 542.6 | 1003.4 | 1054.1 | | | | | | | | |
| 1253 r | 100.0 | 136.9 | 259.2 | 266.3 | 419.0 | 482.9 | 505.1 | >21 | 100.0 | 101.5 | 102.6 | 102.2 | 103.7 | 103.0 | 100.7 |
| 1253 l | 100.0 | 156.9 | 274.4 | 298.9 | 422.2 | 536.0 | 594.3 | | | | | | | | |
| 1279 r | 100.0 | 136.9 | 198.3 | 341.7 | 497.4 | 623.2 | 825.2 | >21 | 100.0 | 99.6 | 101.8 | 100.7 | 102.8 | 102.5 | 102.1 |
| 1279 l | 100.0 | 120.9 | 162.3 | 221.2 | 525.5 | 575.5 | 605.5 | | | | | | | | |
| n | 20 | 20 | 20 | 20 | 20 | 20 | 20 | | 10 | 10 | 10 | 10 | 10 | 10 | 10 |
| Median | 100.0 | 129.7 | 217.8 | 288.5 | 507.0 | 628.1 | 745.4 | | 100.0 | 101.9 | 105.2 | 106.4 | 108.1 | 106.4 | 105.2 |
| MIN | 100.0 | 100.0 | 152.5 | 213.3 | 323.4 | 386.8 | 450.4 | | 100.0 | 98.1 | 98.8 | 97.3 | 98.1 | 96.1 | 90.1 |
| MAX | 100.0 | 190.7 | 380.9 | 490.5 | 964.1 | 1339.8 | 1471.2 | | 100.0 | 105.2 | 108.3 | 109.7 | 113.7 | 115.2 | 113.7 |
| 95% MIN | 100.0 | 124.8 | 205.2 | 278.0 | 497.4 | 623.2 | 685.4 | | 100.0 | 101.5 | 102.6 | 102.2 | 103.7 | 102.5 | 100.7 |
| 95% MAX | 100.0 | 146.1 | 250.0 | 341.7 | 612.8 | 829.8 | 899.6 | | 100.0 | 103.2 | 105.3 | 107.6 | 110.3 | 107.4 | 108.9 |
| Oncotest Exp-Nr. | H327-2 | | | | Implant/Rando/Induction time | | | | 18 days | | | | | | |
| Tumortyp Nr./Pass | LXFA 1041/9N4 | | | | End result/last study day | | | | 20 | | | | | | |
| Tumor Model | Lung, adeno | | | | Therapy | | | | G-026887; 100 mg/kg/day po; Days 0-20 | | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | 3 | 7 | 10 | 14 | 17 | 20 | [days] | 0 | 3 | 7 | 10 | 14 | 17 | 20 |
| 1258 r | 47.6 | 92.3 | 215.1 | 279.3 | 458.8 | 653.0 | 854.4 | >20 | 25.4 | 25.6 | 25.9 | 25.5 | 25.1 | 24.8 | 25.4 |
| 1258 l | 100.9 | 210.8 | 332.8 | 491.9 | 819.3 | 1083.4 | 1120.0 | | | | | | | | |
| 1261 r | 90.8 | 140.4 | 272.8 | 461.7 | 538.6 | 789.0 | 959.2 | >20 | 28.6 | 29.4 | 30.9 | 30.4 | 30.7 | 30.3 | 31.4 |
| 1261 l | 111.6 | 209.5 | 291.2 | 420.1 | 575.5 | 859.6 | 973.4 | | | | | | | | |
| 1262 r | 68.8 | 139.3 | 258.8 | 284.6 | 510.9 | 853.9 | 925.7 | >20 | 24.5 | 24.6 | 25.2 | 24.9 | 24.8 | 25.1 | 25.8 |
| 1262 l | 57.6 | 90.8 | 147.9 | 237.2 | 401.6 | 629.7 | 665.5 | | | | | | | | |
| 1267 r | 62.5 | 68.8 | 212.5 | 326.4 | 672.9 | 1197.5 | 1422.1 | >20 | 24.5 | 23.8 | 24.3 | 24.3 | 24.6 | 25.0 | 26.3 |
| 1267 l | 90.8 | 145.4 | 294.4 | 510.3 | 1044.8 | 1567.4 | 1661.1 | | | | | | | | |
| 1269 r | 84.7 | 117.0 | 197.0 | 274.4 | 426.3 | 790.0 | 906.6 | >20 | 24.0 | 23.6 | 24.4 | 23.8 | 24.4 | 24.7 | 26.1 |
| 1269 l | 50.6 | 98.3 | 126.0 | 173.2 | 312.1 | 471.5 | 639.9 | | | | | | | | |
| 1270 r | 210.8 | 292.8 | 470.6 | 701.8 | 1321.4 | 1975.9 | 2047.5 | >20 | 25.1 | 25.1 | 25.4 | 25.5 | 26.1 | 26.2 | 28.3 |
| 1270 l | 117.2 | 201.6 | 344.4 | 451.3 | 613.8 | 974.7 | 1008.0 | | | | | | | | |
| 1273 r | 139.4 | 249.4 | 392.9 | 546.2 | 1022.5 | 1193.5 | 1366.9 | >20 | 29.8 | 29.3 | 29.8 | 30.0 | 31.2 | 31.9 | 33.2 |
| 1273 l | 86.2 | 159.5 | 180.3 | 228.1 | 451.2 | 643.5 | 853.1 | | | | | | | | |
| 1275 r | 62.5 | 98.3 | 164.8 | 264.7 | 635.1 | 949.9 | 1064.6 | >21 | 31.5 | 30.4 | 30.5 | 30.6 | 30.4 | 31.1 | 32.4 |
| 1275 l | 75.7 | 120.6 | 169.9 | 258.8 | 402.2 | 520.7 | 605.0 | | | | | | | | |
| 1280 r | 90.8 | 109.3 | 178.9 | 278.4 | 320.3 | 530.5 | 606.4 | >21 | 25.0 | 25.4 | 26.3 | 26.1 | 26.8 | 26.4 | 27.4 |
| 1280 l | 81.1 | 120.9 | 170.6 | 258.8 | 432.7 | 642.0 | 755.2 | | | | | | | | |
| 1281 r | 117.0 | 199.6 | 240.9 | 465.8 | 617.9 | 687.3 | 931.6 | >21 | 23.6 | 22.9 | 22.6 | 22.0 | 22.7 | 22.7 | 23.1 |
| 1281 l | 123.0 | 225.0 | 285.8 | 405.0 | 544.0 | 661.5 | 790.1 | | | | | | | | |
| n | 20 | 20 | 20 | 20 | 20 | 20 | 20 | | 10 | 10 | 10 | 10 | 10 | 10 | 10 |
| Median | 88.5 | 139.8 | 228.0 | 305.5 | 541.3 | 789.5 | 928.6 | | 25.1 | 25.3 | 25.7 | 25.5 | 25.6 | 25.7 | 26.9 |
| MIN | 47.6 | 68.8 | 126.0 | 173.2 | 312.1 | 471.5 | 605.0 | | 23.6 | 22.9 | 22.6 | 22.0 | 22.7 | 22.7 | 23.1 |
| MAX | 210.8 | 292.8 | 470.6 | 701.8 | 1321.4 | 1975.9 | 2047.5 | | 31.5 | 30.4 | 30.9 | 30.6 | 31.2 | 31.9 | 33.2 |
| 95% MIN | 81.1 | 139.3 | 212.5 | 326.4 | 510.9 | 789.0 | 853.1 | | 24.5 | 24.6 | 25.2 | 24.3 | 24.6 | 24.7 | 25.8 |
| 95% MAX | 100.9 | 159.5 | 285.8 | 420.1 | 672.9 | 974.7 | 1120.0 | | 25.4 | 25.6 | 26.3 | 26.1 | 26.8 | 26.4 | 28.3 |
| | Relative tumor volume [%] | | | | | | | | Relative body weight [%] | | | | | | |
| Animal-No | 0 | 3 | 7 | 10 | 14 | 17 | 20 | | 0 | 3 | 7 | 10 | 14 | 17 | 20 |
| 1258 r | 100.0 | 193.9 | 452.1 | 586.9 | 964.1 | 1372.2 | 1795.5 | >20 | 100.0 | 100.8 | 102.0 | 100.4 | 98.8 | 97.6 | 100.0 |
| 1258 l | 100.0 | 208.9 | 329.8 | 487.4 | 811.9 | 1073.6 | 1109.8 | | | | | | | | |
| 1261 r | 100.0 | 154.7 | 300.6 | 508.8 | 593.5 | 869.4 | 1056.9 | >20 | 100.0 | 102.8 | 108.0 | 106.3 | 107.3 | 105.9 | 109.8 |
| 1261 l | 100.0 | 187.7 | 260.9 | 376.4 | 515.7 | 770.2 | 872.2 | | | | | | | | |
| 1262 r | 100.0 | 202.6 | 376.4 | 414.0 | 743.1 | 1242.1 | 1346.4 | >20 | 100.0 | 100.4 | 102.9 | 101.6 | 101.2 | 102.4 | 105.3 |
| 1262 l | 100.0 | 157.6 | 256.7 | 411.7 | 697.3 | 1093.2 | 1155.4 | | | | | | | | |
| 1267 r | 100.0 | 110.0 | 340.1 | 522.2 | 1076.6 | 1915.9 | 2275.4 | >20 | 100.0 | 97.1 | 99.2 | 99.2 | 100.4 | 102.0 | 107.3 |
| 1267 l | 100.0 | 160.2 | 324.4 | 562.3 | 1151.3 | 1727.1 | 1830.4 | | | | | | | | |
| 1269 r | 100.0 | 138.1 | 232.6 | 323.9 | 503.3 | 932.7 | 1070.4 | >20 | 100.0 | 98.3 | 101.7 | 99.2 | 101.7 | 102.9 | 108.8 |
| 1269 l | 100.0 | 194.2 | 248.9 | 342.2 | 616.5 | 931.4 | 1264.0 | | | | | | | | |
| 1270 r | 100.0 | 138.9 | 223.2 | 332.9 | 626.8 | 937.2 | 971.2 | >20 | 100.0 | 100.0 | 101.2 | 101.6 | 104.0 | 104.4 | 112.7 |
| 1270 l | 100.0 | 172.0 | 293.8 | 385.0 | 523.7 | 831.6 | 860.0 | | | | | | | | |
| 1273 r | 100.0 | 178.9 | 281.8 | 391.8 | 733.4 | 856.0 | 980.4 | >20 | 100.0 | 98.3 | 100.0 | 100.7 | 104.7 | 107.0 | 111.4 |
| 1273 l | 100.0 | 185.0 | 209.2 | 264.6 | 523.3 | 746.4 | 989.5 | | | | | | | | |
| 1275 r | 100.0 | 157.3 | 263.6 | 423.4 | 1016.2 | 1519.8 | 1703.4 | >21 | 100.0 | 96.5 | 96.8 | 97.1 | 96.5 | 98.7 | 102.9 |
| 1275 l | 100.0 | 159.3 | 224.4 | 341.8 | 531.2 | 687.7 | 799.1 | | | | | | | | |
| 1280 r | 100.0 | 120.5 | 197.1 | 306.8 | 353.0 | 584.5 | 668.2 | >21 | 100.0 | 101.6 | 105.2 | 104.4 | 107.2 | 105.6 | 109.6 |
| 1280 l | 100.0 | 149.1 | 210.3 | 319.0 | 533.4 | 791.4 | 931.0 | | | | | | | | |
| 1281 r | 100.0 | 170.6 | 205.9 | 398.1 | 528.1 | 587.5 | 796.3 | >21 | 100.0 | 97.0 | 95.8 | 93.2 | 96.2 | 96.2 | 97.9 |
| 1281 l | 100.0 | 182.9 | 232.3 | 329.2 | 442.2 | 537.8 | 642.3 | | | | | | | | |
| n | 20 | 20 | 20 | 20 | 20 | 20 | 20 | | 10 | 10 | 10 | 10 | 10 | 10 | 10 |
| Median | 100.0 | 165.4 | 258.8 | 388.4 | 605.0 | 900.4 | 1023.2 | | 100.0 | 99.2 | 101.4 | 100.5 | 101.4 | 102.7 | 108.0 |
| MIN | 100.0 | 110.0 | 197.1 | 264.6 | 353.0 | 537.8 | 642.3 | | 100.0 | 96.5 | 95.8 | 93.2 | 96.2 | 96.2 | 97.9 |
| MAX | 100.0 | 208.9 | 452.1 | 586.9 | 1151.3 | 1915.9 | 2275.4 | | 100.0 | 102.8 | 108.0 | 106.3 | 107.3 | 107.0 | 112.7 |
| 95% MIN | 100.0 | 154.7 | 248.9 | 376.4 | 593.5 | 831.6 | 971.2 | | 100.0 | 98.3 | 99.2 | 99.2 | 100.4 | 102.0 | 105.3 |
| 95% MAX | 100.0 | 172.0 | 300.6 | 423.4 | 743.1 | 1093.2 | 1346.4 | | 100.0 | 100.8 | 102.9 | 101.6 | 104.0 | 104.4 | 109.8 |
| Oncotest Exp-Nr. | H327-3 | | | | Implant/Rando/Induction time | | | | 18 days | | | | | | |
| Tumortyp Nr./Pass | LXFA 1041/9N4 | | | | End result/last study day | | | | 20 | | | | | | |
| Tumor Model | Lung, adeno | | | | Therapy | | | | None | | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | | | | | | | [days] | | | | 0 | | | |
| 1251 r | 77.2 | | | | | | | >0 | | | | 26.2 | | | |
| 1254 r | 95.8 | | | | | | | >0 | | | | 20.8 | | | |
| 1264 l | 93.8 | | | | | | | >0 | | | | 23.0 | | | |
| n | 3 | | | | | | | | | | | 3 | | | |
| Median | 93.8 | | | | | | | | | | | 23.0 | | | |
| MIN | 77.2 | | | | | | | | | | | 20.8 | | | |
| MAX | 95.8 | | | | | | | | | | | 26.2 | | | |
| 95% MIN | 77.2 | | | | | | | | | | | 20.8 | | | |
| 95% MAX | 95.8 | | | | | | | | | | | 26.2 | | | |
| | Relative tumor volume [%] | | | | | | | | Relative body weight [%] | | | | | | |
| Animal-No | 0 | | | | | | | | | | | 0 | | | |
| 1251 r | 100.0 | | | | | | | >0 | | | | 100.0 | | | |
| 1254 r | 100.0 | | | | | | | >0 | | | | 100.0 | | | |
| 1264 l | 100.0 | | | | | | | >0 | | | | 100.0 | | | |
| n | 3 | | | | | | | | | | | 3 | | | |
| Median | 100.0 | | | | | | | | | | | 100.0 | | | |
| MIN | 100.0 | | | | | | | | | | | 100.0 | | | |
| MAX | 100.0 | | | | | | | | | | | 100.0 | | | |
| 95% MIN | 100.0 | | | | | | | | | | | 100.0 | | | |
| 95% MAX | 100.0 | | | | | | | | | | | 100.0 | | | |
| Oncotest Exp-Nr. | H327-4 | | | | Implant/Rando/Induction time | | | | 18 days | | | | | | |
| Tumortyp Nr./Pass | LXFA 1041/9N4 | | | | End result/last study day | | | | 20 | | | | | | |
| Tumor Model | Lung, adeno | | | | Therapy | | | | Vehicle control; 10 ml/kg; Day 0 | | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization | | | | | | |
| Animal-No | 0 | | | | | | | [days] | | | | 0 | | | |
| 1239 r | 344.6 | | | | | | | >0 | | | | 27.9 | | | |
| 1246 l | 287.6 | | | | | | | >0 | | | | 24.0 | | | |
| 1255 r | 216.3 | | | | | | | >0 | | | | 24.7 | | | |
| 1260 r | 425.3 | | | | | | | >0 | | | | 27.2 | | | |
| 1266 l | 183.8 | | | | | | | >0 | | | | 27.5 | | | |
| 1268 r | 200.9 | | | | | | | >0 | | | | 26.0 | | | |
| n | 6 | | | | | | | | | | | 6 | | | |
| Median | 251.9 | | | | | | | | | | | 26.6 | | | |
| MIN | 183.8 | | | | | | | | | | | 24.0 | | | |
| MAX | 425.3 | | | | | | | | | | | 27.9 | | | |
| 95% MIN | 183.8 | | | | | | | | | | | 24.7 | | | |
| 95% MAX | 344.6 | | | | | | | | | | | 27.5 | | | |
| | Relative tumor volume [%] | | | | | | | | Relative body weight [%] | | | | | | |
| Animal-No | 0 | | | | | | | | | | | 0 | | | |
| 1239 r | 100.0 | | | | | | | >0 | | | | 100.0 | | | |
| 1246 l | 100.0 | | | | | | | >0 | | | | 100.0 | | | |
| 1255 r | 100.0 | | | | | | | >0 | | | | 100.0 | | | |
| 1260 r | 100.0 | | | | | | | >0 | | | | 100.0 | | | |
| 1266 l | 100.0 | | | | | | | >0 | | | | 100.0 | | | |
| 1268 r | 100.0 | | | | | | | >0 | | | | 100.0 | | | |
| n | 6 | | | | | | | | | | | 6 | | | |
| Median | 100.0 | | | | | | | | | | | 100.0 | | | |
| MIN | 100.0 | | | | | | | | | | | 100.0 | | | |
| MAX | 100.0 | | | | | | | | | | | 100.0 | | | |
| 95% MIN | 100.0 | | | | | | | | | | | 100.0 | | | |
| 95% MAX | 100.0 | | | | | | | | | | | 100.0 | | | |
| Oncotest Exp-Nr. | H327-5 | | | | Implant/Rando/Induction time | | | | 18 days | | | | | | |
| Tumortyp Nr./Pass | LXFA 1041/9N4 | | | | End result/last study day | | | | 20 | | | | | | |
| Tumor Model | Lung, adeno | | | | Therapy | | | | G-026887; 100 mg/kg/day po; Day 0 | | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | [days] | 0 | | | | | [days] | | | | 0 | | | |
| 1257 r | 256.0 | | | | | | | >0 | | | | 26.3 | | | |
| 1263 l | 219.0 | | | | | | | >0 | | | | 23.5 | | | |
| 1271 l | 423.2 | | | | | | | >0 | | | | 24.9 | | | |
| 1274 r | 441.1 | | | | | | | >0 | | | | 31.1 | | | |
| 1276 r | 166.5 | | | | | | | >0 | | | | 20.8 | | | |
| 1277 r | 240.1 | | | | | | | >0 | | | | 27.4 | | | |
| n | 6 | | | | | | | | | | | 6 | | | |
| Median | 248.1 | | | | | | | | | | | 25.6 | | | |
| MIN | 166.5 | | | | | | | | | | | 20.8 | | | |
| MAX | 441.1 | | | | | | | | | | | 31.1 | | | |
| 95% MIN | 219.0 | | | | | | | | | | | 23.5 | | | |
| 95% MAX | 256.0 | | | | | | | | | | | 27.4 | | | |
| | Relative tumor volume [%] | | | | | | | | relative body weight [%] | | | | | | |
| Animal-No | 0 | | | | | | | | | | | 0 | | | |
| 1257 r | 100.0 | | | | | | | >0 | | | | 100.0 | | | |
| 1263 l | 100.0 | | | | | | | >0 | | | | 100.0 | | | |
| 1271 l | 100.0 | | | | | | | >0 | | | | 100.0 | | | |
| 1274 r | 100.0 | | | | | | | >0 | | | | 100.0 | | | |
| 1276 r | 100.0 | | | | | | | >0 | | | | 100.0 | | | |
| 1277 r | 100.0 | | | | | | | >0 | | | | 100.0 | | | |
| n | 6 | | | | | | | | | | | 6 | | | |
| Median | 100.0 | | | | | | | | | | | 100.0 | | | |
| MIN | 100.0 | | | | | | | | | | | 100.0 | | | |
| MAX | 100.0 | | | | | | | | | | | 100.0 | | | |
| 95% MIN | 100.0 | | | | | | | | | | | 100.0 | | | |
| 95% MAX | 100.0 | | | | | | | | | | | 100.0 | | | |
| Oncotest Exp-Nr. | H327-6 | | | | Implant/Rando/Induction time | | | | 18 days | | | | | | |
| Tumortyp Nr./Pass | LXFA 1041/9N4 | | | | End result/last study day | | | | 20 | | | | | | |
| Tumor Model | Lung, adeno | | | | Therapy | | | | G-026887; 100 mg/kg/day po; Day 0 | | | | | | |
| | Absolute tumor volume: (a*b*b)/2 [mm3] | | | | | | | Survival | Absolute body weight [g] | | | | | | |
| | Study day (after randomization) | | | | | | | time | Study day (after randomization) | | | | | | |
| Animal-No | 0 | | | | | | | [days] | | | | 0 | | | |
| 1240 r | 409.1 | | | | | | | >0 | | | | 27.9 | | | |
| 1241 r | 250.5 | | | | | | | >0 | | | | 27.2 | | | |
| 1256 l | 267.7 | | | | | | | >0 | | | | 25.2 | | | |
| n | 3 | | | | | | | | | | | 3 | | | |
| Median | 267.7 | | | | | | | | | | | 27.2 | | | |
| MIN | 250.5 | | | | | | | | | | | 25.2 | | | |
| MAX | 409.1 | | | | | | | | | | | 27.9 | | | |
| 95% MIN | 250.5 | | | | | | | | | | | 25.2 | | | |
| 95% MAX | 409.1 | | | | | | | | | | | 27.9 | | | |
| | Relative tumor volume [%] | | | | | | | | Relative body weight [%] | | | | | | |
| Animal-No | 0 | | | | | | | | | | | 0 | | | |
| 1240 r | 100.0 | | | | | | | >0 | | | | 100.0 | | | |
| 1241 r | 100.0 | | | | | | | >0 | | | | 100.0 | | | |
| 1256 l | 100.0 | | | | | | | >0 | | | | 100.0 | | | |
| n | 3 | | | | | | | | | | | 3 | | | |
| Median | 100.0 | | | | | | | | | | | 100.0 | | | |
| MIN | 100.0 | | | | | | | | | | | 100.0 | | | |
| MAX | 100.0 | | | | | | | | | | | 100.0 | | | |
| 95% MIN | 100.0 | | | | | | | | | | | 100.0 | | | |
| 95% MAX | 100.0 | | | | | | | | | | | 100.0 | | | |

What is claimed is:

1. A method of identifying a patient nonresponsive to treatment with a B-Raf inhibitor, comprising determining the presence or absence of a K-ras mutation, whereby the presence of a K-ras mutation indicates a patient will not respond to said B-Raf inhibitor treatment.

2. A method of determining whether a tumor will respond to treatment with a B-Raf inhibitor, comprising determining in a sample of said tumor the presence of a mutant K-ras protein or gene whereby the presence of a mutant K-ras protein or gene indicates that the tumor will not respond to treatment with a B-Raf inhibitor.

3. The method of claim 1 wherein said K-ras mutation is an activating mutation.

4. The method of claim 1 wherein said K-ras mutation is at least one of G12C; G12A; G12D; G12R; G12S; G12V; G13C; and G13D.

5. The method of claim 1 wherein said B-Raf inhibitor is a specific B-Raf kinase inhibitor.

6. The method of claim 1 wherein the presence of a K-ras mutation is determined by amplifying K-ras nucleic acid from said tumor, or a fragment thereof suspected of containing a mutation, and sequencing said amplified nucleic acid.

7. The method of claim 1 wherein the presence of a K-ras mutation is determined by amplifying K-RAS nucleic acid from said tumor, or a fragment thereof suspected of containing a mutation, and comparing the electrophoretic mobility of the amplified nucleic acid to the electrophoretic mobility of corresponding wild-type K-ras nucleic acid or fragment.

8. A method of predicting whether a patient will be nonresponsive to treatment with a specific B-Raf inhibitor, comprising determining the presence or absence of a K-ras mutation in a tumor of the patient, wherein the K-ras mutation is in codon 12 or codon 13; and wherein if a K-ras mutation is present, the patient is predicted to be nonresponsive to treatment with a specific B-Raf inhibitor.

9. The method of claim 1, wherein the determining the presence or absence of a K-ras mutation in a tumor comprises amplifying a K-ras nucleic acid from the tumor and sequencing the amplified nucleic acid.

10. The method of claim 1, wherein the determining the presence or absence of a K-ras mutation in a tumor comprises detecting a mutant K-ras polypeptide in a sample of the tumor using a specific binding agent to a mutant K-ras polypeptide.

11. The method of claim 1, wherein the K-ras mutation is selected from G12S, G12V, G12D, G12A, G12C, G13A, and G13D.

12. A kit useable for the method of claim 1, comprising material specific for detecting a K-ras mutant gene or protein, and material specific for detecting a B-Raf mutant gene or protein.

13. The kit of claim 12, further comprising instructions for use in identifying a patient nonresponsive to treatment with a B-Raf inhibitor.

14. The kit of claim 13, wherein said patient has lung or colorectal cancer.

15. A method of classifying a breast, lung, colon, ovarian, thyroid, melanoma or pancreatic tumor comprising the steps of: obtaining a tumor sample; detecting expression or activity of a (i) a gene encoding the B-Raf V600E mutant and (ii) a gene encoding a k-Ras mutant in the sample.

16. The method of claim 15, further comprising classifying the tumor as belonging to a tumor subclass based on the results of the detecting step; and selecting a treatment based on the classifying step, wherein said treatment is other than a B-Raf V600E specific inhibitor if said k-RAS mutant is overexpressed in said tumor sample.

17. A method of identifying a tumor nonresponsive to treatment with a B-Raf inhibitor, comprising determining the expression level of a receptor tyrosine kinase (RTK), whereby aberrant expression or induction of said RTK indicates said patient will not respond to said B-Raf inhibitor treatment, and wherein said tumor expresses B-Raf V600E.

18. The method of claim 17, wherein said RTK is EGFR or cMET.

19. The method of claim 18, further comprising treating said tumor by administering an effective amount of an inhibitor of said EGFR or cMET in combination with a B-Raf inhibitor.

20. The method of claim 19, wherein said EGFR inhibitor is erlotinib.

21. The method of claim 20, wherein said combination is administered in a synergistic amount.

22. The method of claim 17, wherein said tumor type is colon or melanoma.
